(19) United States
(12) Patent Application Publication
(10) Pub. No.: US 2015/0151094 A1
(43) Pub. Date: Jun. 4, 2015

(54) Devices for Maintaining An Oropharyngeal Airway, Methods of Creating An Oropharyngeal Airway and Systems for Mainitaining An Oropharyngeal Airway

(71) Applicant: Brian LEWER, Doonan, Queensland (AU)

(72) Inventor: Brian Lewer, Doonan (AU)

(21) Appl. No.: 14/386,562

(22) PCT Filed: Mar. 20, 2013
(86) PCT No.: PCT/AU2013/000287

§ 371 Date: Sep. 19, 2014

(30) Foreign Application Priority Data
Mar. 20, 2012 (AU) 2012901116

(51) Int. Cl. A61M 29/02; A61M 16/04

(52) U.S. Cl. CPC A61M 29/02; A61M 16/0409; A61M 16/0461; A61M 2029/025; A61M 16/0434

(57) ABSTRACT

An oropharyngeal airway device and method, including inserting a nasopharyngeal airway means in a collapsed configuration through one of the nares, nasal cavity and nasopharynx and into the pharynx to locate an expandable body in a collapsed configuration in the pharynx substantially around the glottis, epiglottis and laryngeal opening; and expanding the nasopharyngeal airway means and the expandable body from the collapsed configuration to an expanded configuration to create the oropharyngeal airway through the nasopharyngeal airway means and expandable body.

### FIELD OF THE INVENTION

The present invention relates to devices for maintaining an oropharyngeal airway, methods of creating an oropharyngeal airway and systems for maintaining an oropharyngeal airway.

The invention has been developed primarily for use in treating obstructive apnea and for airway management during sedation or anesthesia and will be described hereinafter with reference to these applications. However, it will be appreciated that the invention is not limited to these particular fields of use.

### BACKGROUND OF THE INVENTION

Apnea and Hypopnea

Obstructive sleep apnea (OSA) is a sleep-related breathing disorder that can cause a person to experience periods of apnea and hypopnea during sleep. Apnea is defined as the cessation of breathing for a period ten seconds or more, and hypopnea as the reduction in airflow by more than 50% for a period of ten seconds or more.

Obstructive sleep apnea most commonly occurs due to the loss of muscle tone during sleep which causes the tongue and/or soft tissue in the throat to collapse and narrow or obstruct the pharyngeal airway. This is most likely to occur in the oropharyngeal region of the airway due to a closure of the space between the posterior pharyngeal wall and the tongue, which occludes both nasal and oral airflow. The obstruction can also occur in the nasopharyngeal/soft palate region of the airway due to a closure of the space between the posterior pharyngeal wall and the soft palate, occluding nasal airflow. In obstructive sleep apnea, although respiratory effort continues, respiration cannot occur due to the obstruction of the airway. This results in oxygen desaturation and arousals from sleep.

OSA affects 24% of men and 9% of women in the general population, and often causes excessive sleepiness, unrefreshing sleep, poor concentration, mental health problems and fatigue during the day. If untreated, OSA has been shown to cause increased incidence of coronary heart disease, hypertension, congestive heart failure, cerebrovascular accidents, gastroesophageal reflux disease and decreased life expectancy.

Apnea and hypopnea can also occur during sedation or anesthesia, perioperatively, when decreased muscle tone causes the upper airways to collapse. It has been found that difficult intubation is eight times more likely to occur in patients suffering from major morbidity and obstructive sleep apnea than in patients who did not suffer from OSA. Furthermore, 30% of anesthesia-related mortality and morbidity is caused by the loss of airway patency. It is therefore extremely important to monitor and manage the patient's airway during anesthesia and sedation.

Treatment Available for OSA

The two main classes of devices developed to treat OSA are positive airway pressure (PAP) devices, and oral appliances. PAP is the application of positive pressure to the airway to pneumatically splint the pharyngeal airway, thus preventing upper airway collapse. The different forms of PAP include continuous PAP, bi-level PAP, autotitrating PAP, and adaptive servoventilation. The most commonly administered form is continuous PAP, or CPAP.

CPAP is typically provided by using a pressurised mask connected to a device that delivers positive pressure. Common adverse effects of CPAP include nasal congestion, nasal dryness, eye irritation due to mask leaks, abrasion of the bridge of the nose, dermatitis and tinnitus. Adherence to CPAP therapy can also be a problem because of:
mask interference issues, which might, for example, inhibit or preclude the freedom of movement of the patient during sleep,
a feeling of claustrophobia,
nasal congestion,
machine noise,
pressure intolerance, or
a combination of the above.

Oral appliances take the form of either mandibular advancement splints, which maintain the mandible in a protruded position during sleep, or tongue stabilising devices, which maintain the tongue in a protruded position during sleep. These oral appliances are more likely to be successful for patients with mild to moderate OSA, however, can cause excess salivation, headaches, and temporomandibular joint symptoms.

Surgical techniques are also available to treat OSA. These techniques include maxilla-mandibular advancement (MMA), uvulopalatopharyngoplasty (UPPP), upper airway radiofrequency ablation and tracheostomy. However, surgical treatment is invasive, and is typically only pursued if the patient has a strong preference or an intolerance to PAP.

The present invention seeks to provide a supraglottic airway device, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

Oropharyngeal Cushion

According to a first aspect of the present invention, a device for maintaining an oropharyngeal airway is provided, comprising:
a body adapted for configuration between a collapsed and an expanded configuration,
wherein in the expanded configuration, the body is adapted for location in the oropharynx, to define an oropharyngeal airway and to maintain the oropharyngeal airway at least sufficiently open for breathing when the body is subjected to an external pressure,
the oropharyngeal airway being substantially larger when the body is in the expanded configuration than when the body is in the collapsed configuration.

Advantageously, the device for maintaining an oropharyngeal airway can be used for the treatment of obstructive sleep apnea.

Advantageously, the device for maintaining an oropharyngeal airway can be used with all forms of PAP including during anesthesia and sedation.

Advantageously, the device for maintaining an oropharyngeal airway can be used in a surgical context for securing the patency of the patient's airway perioperatively, while the patient is anaesthetized or sedated, or postoperatively.

Pressure

Preferably, the external pressure is applied by the base of the tongue.

Advantageously, the device for maintaining an oropharyngeal airway is effective in resisting the collapse of the base of the tongue. As with apnea and hypopnea during sleep, anesthesia or sedation can be caused by the collapse of the base of the tongue into the oropharynx causing obstruction of the airway, the device is therefore effective in maintaining the oropharyngeal airway at least sufficiently open in use so that the patient is able to continue breathing.

Preferably, the external pressure is applied by the oropharyngeal wall.

Advantageously, the device for maintaining an oropharyngeal airway is effective in resisting the collapse of the oropharyngeal wall and relaxation of the tongue muscles. As with apnea and hypopnea during sleep, anesthesia or sedation can be caused by the collapse of the oropharyngeal wall into the oropharynx causing obstruction of the airway, the device is therefore effective in maintaining the oropharyngeal airway at least sufficiently open in use so that the patient is able to continue breathing.

Preferably, in the expanded configuration, the body is adapted to apply a pressure to one or more areas from the following group of areas:
the oropharyngeal wall;
the posterior pharyngeal wall;
the base of the tongue; and
the soft palate.

Advantageously, the device for maintaining an oropharyngeal airway is effective in resisting the collapse of one or a combination of the oropharyngeal wall, the posterior pharyngeal wall, the base of the tongue and the soft palate. As apnea and hypopnea during sleep, anesthesia or sedation can be caused by the collapse of one or a combination of the abovementioned tissues into the oropharynx, causing obstruction of the airway, the device is therefore effective in maintaining the oropharyngeal airway at least sufficiently open in use so that the patient is able to continue breathing.

Location

Preferably, the body is adapted for location at a location upstream of the cartilaginous epiglottis.

As the oropharyngeal region of the patient's natural airway can become obstructed and cause apnea or hypopnea, it is advantageous that the body is adapted for location in the oropharyngeal region and is therefore effective in maintaining the patient's airway at least sufficiently open for breathing in use.

Preferably, at the location, the body is not in contact with the cartilaginous epiglottis.

It is advantageous that the body in use does not interfere with the function of the epiglottis and that the body is not displaced from its position within the oropharynx by movement of the epiglottis.

Size & Shape

Preferably, the body has a length of between 10 and 40 mm.

Preferably, the body has a length of between 20 and 35 mm.

Preferably, the body has a length of between 25 and 30 mm.

Preferably, in the expanded configuration, the body has a maximum width of between 5 and 12 mm.

Preferably, in the expanded configuration, the body has a maximum width of between 6 and 10 mm.

Advantageously, the body in the expanded configuration is adapted to comfortably and snugly fit into the oropharyngeal region of the patient's airway, and to provide sufficient pressure against the collapsible tissues of the patient's airway to maintain the airway at least sufficiently open for breathing.

Preferably, in the collapsed configuration, the body fits within a diameter of 10 mm.

Preferably, in the collapsed configuration, the body fits within a diameter of 7 mm.

Preferably, in the collapsed configuration, the body fits within a diameter of about 6 mm.

Advantageously, minimizing the size of the body in the collapsed configuration improves the ease of maneuverability of the body into the patient's natural airway, thus minimizing the discomfort experienced by the patient during the insertion procedure. Once located in the desired position within the patient's natural airway, the body can then be deployed into the expanded configuration to define the oropharyngeal airway.

Preferably, the oropharyngeal airway has a minimum cross-sectional area of between 6 and 13 mm when the body is in the expanded configuration.

Preferably, the oropharyngeal airway has a minimum cross-sectional area of between 7 and 9 mm when the body is in the expanded configuration.

Advantageously, the oropharyngeal airway is sufficiently large in use to facilitate breathing with minimal resistance. This will allow the patient to breathe naturally and comfortably, which, if the device is used for treatment of OSA, encourages compliance.

Preferably, in the expanded configuration, the body is generally torus shaped.

Advantageously, a torus shape is a shape that defines an internal passageway, the passageway forming the oropharyngeal airway.

Preferably, in the expanded configuration, the body has an external diameter of between 5 mm and 12 mm.

Preferably, the external diameter is between 6 mm and 10 mm.

Advantageously, the body in the expanded configuration is adapted to comfortably and snugly fit into the oropharyngeal region of a patient's airway, and to provide sufficient pressure against the collapsible tissues of the patient's airway to maintain the airway at least sufficiently open for breathing.

Preferably, in the expanded configuration, the body has an internal diameter of between 4 mm and 9 mm.

Preferably, the internal diameter is between 4 mm and 8 mm.

Preferably, the body is a generally thin-walled structure.

Advantageously, the body is proportioned such that the cross-sectional area of the oropharyngeal airway is maximized to facilitate breathing with minimal resistance in use. Nevertheless, the body is adapted to sufficiently withstand the application of the external pressure from the collapsible tissues of the patient's airway in use.

Preferably, the device for maintaining an oropharyngeal airway comprises one or more fold lines to control movement of the body between the expanded and the collapsed configuration.

Advantageously, the body is easily and reliably configurable between the collapsed and expanded configuration in use.

Preferably, when the body is in the collapsed configuration it is twisted.

Preferably, when the body is in the expanded configuration, the oropharyngeal airway comprises an opening towards the nasopharynx and another opening towards the mouth and tongue.

Advantageously, the body facilitates both breathing through the nose and through the mouth.

Preferably, the body is sized to be received within a pediatric patient.

Preferably, the body is sized to be received within an adult patient.

Advantageously, the device can be produced in various sizes to fit both adults and children.

Guide Means

Preferably, the device for maintaining an oropharyngeal airway further comprises guide means connected to the body.

Preferably, the guide means is a nasopharyngeal guide means adapted to direct the body in the collapsed configuration through one of the nares, nasal cavity and nasopharynx and into the pharynx.

Advantageously, the body is easily and readily inserted and directed into the desired position in the patient's pharynx. If correctly inserted, the patient may feel minimal discomfort during and following the insertion procedure, and will not likely sustain any damage to the nares, nasal cavity, nasopharynx or pharynx. Insofar as the device for maintaining an oropharyngeal airway is used for treating patients suffering from obstructive sleep apnea, the ease and comfort with which the body can be inserted facilitates compliance by the patient in self-administration of the treatment. Moreover, the device can be easily directed into place by a person who is not a medical practitioner, including the patient himself or herself, and is thus suitable for regular home use.

Advantageously, the device for maintaining an oropharyngeal airway allows the intake of air into the patient's airways to be naturally humidified and filtered by the patient's own nasal passages and/or oral passage in use.

Advantageously, insertion of the device and the location of the device in situ are relatively comfortable and well-tolerated, allowing the device to be inserted while the patient is awake. This allows the patient's airway to be secured prior to the induction of anesthesia or sedation, reducing the likelihood of difficult intubation, and of anesthesia-related mortality and morbidity caused by loss of the patency of the airway.

Advantageously, as the device is adapted to access the patient's airway through the nasal passages, rather than through the oral passage, the patient's airway can be secured during maxillofacial or oral procedures while still allowing perfect oral access.

Preferably, the guide means is adapted to direct the body such that it does not apply a substantial pressure to the region of the turbinate at the entrance to the nasopharynx.

Preferably, the guide means is adapted to direct the body such that it does not contact the region of the turbinate at the entrance to the nasopharynx.

Advantageously, as the region of the turbinate is a sensitive region, the body can be inserted and directed into place without the patient experiencing excessive discomfort or suffering damage in the region of the turbinate.

Preferably, the guide means is connected to a region of the body that is posterior to the oropharyngeal airway in use when the body is in the expanded configuration.

Advantageously, the guide means does not interfere with, narrow, or in any way restrict the oropharyngeal airway in use.

Preferably, the guide means is flexible.

Preferably, wherein the guide means is semi-rigid.

Preferably, the guide means is pliable.

Preferably, the length of the guide means is bendable into different shapes.

Advantageously, the guide means is adapted to facilitate ease of insertion of the body into the pharynx.

Preferably, the guide means comprises a thermo-sensitive material such that at least one material property of the thermo-sensitive material changes as a function of temperature.

Preferably, the thermo-sensitive material changes shape as a function of temperature such that the guide means changes shape as a function of temperature.

Advantageously, the shape of the guide means is be adapted to change shape after insertion such that it better conforms to the natural contours of the nares, nasal cavity and nasopharynx of the patient for greater comfort in use.

Preferably, the guide means comprises a shape memory material and is bendable into a new shape when heated and retains the new shape when cooled.

Advantageously, the guide means is be adapted to be tailored to a shape that better conforms to the natural contours of the nares, nasal cavity and nasopharynx of each individual patient, making it more comfortable for the patient in use.

Preferably, the guide means comprises a cord.

Advantageously, the cord may be adapted to be long enough to be located external to the patient's nares in use such that retraction of the device is readily achieved when desired

Preferably, the guide means has an external cross-sectional area of between 0.5 and 3 mm².

Preferably, the guide means has an external cross-sectional area of between 1 and 2 mm².

Advantageously, the guide means is sufficiently narrow such that it is comfortable for the patient and facilitates minimal interference with the breathing of the patient in use, yet facilitates easy guidance of the body into the desired position in the pharynx.

Preferably, the guide means comprises one or more bend regions, each bend region being adapted to move between a first angle and a second angle.

Advantageously, mechanical manipulation of the guide means is limited to a pre-defined range of movement, which prevents the guide means from being bent out of shape or damaged.

Preferably, at least one of the bend regions is adapted to move between the first angle and the second angle upon application of an electric current.

Preferably, the at least one bend region comprises an electro-active shape memory polymer element and is adapted to move between the first angle and the second angle upon application of an electric current to the electro-active shape memory polymer element.

Preferably, the electro-active shape memory polymer element is a piezo-electric polymer element.

Preferably, at least one of the bend regions is adapted to move between the first angle and the second angle upon application of light within a predetermined wavelength range.

Preferably, the at least one bend region comprises a light-induced shape memory polymer element and is adapted to move between the first angle and the second angle upon application of a light within a predetermined wavelength range to the light-induced shape memory polymer element.

Preferably, the at least one bend region comprises an electromechanical servo motor and is adapted to move between the first angle and the second angle upon application of an electric current to the electromechanical servo motor.

Preferably, the guide means comprises a series of elements connected by hinge means, and a tensioning means, the guide means being adapted to move between a first configuration in which the series of elements are disposed relative to each other in a first disposition and a second configuration in which the series of elements are disposed relative to each other in a second disposition by tensioning of the tensioning means.

Preferably, the series of elements are hollow and the tensioning means is a tensioning cord that extends through the series of elements.

Preferably, the guide means comprises a series of elements connected by hinge means, and a push means, the guide means being adapted to move between a first configuration in which the series of elements are disposed relative to each other in a first disposition and a second configuration in which the series of elements are disposed relative to each other in a second disposition by pushing of the push means.

Preferably, the series of elements are hollow and the push means extends through the series of elements.

Preferably, the guide means comprises a sheath means and a rotational member located therein, the rotational member being connected to the body at one end and extending externally from the sheath means at the other end, such that when the rotational member is rotated relative to the sheath means in a first direction the body twists to the collapsed configuration and when the rotational member is rotated relative to the sheath means in the other direction the body un-twists to the expanded configuration.

Preferably, the guide means comprises a sheath means and a push-pull member located therein, the push-pull member being connected to the body at one end and extending externally from the sheath means at the other end, such that when the push-pull member is moved relative to the sheath means in one direction the body collapses to the collapsed configuration and in the other direction the body expands to the expanded configuration.

Advantageously, the guide means can be adapted to be mechanically manipulated by any one or more of a range of different means, to dispose it in a first configuration to optimize it for ease of insertion, and in a second configuration to optimize it for comfort to the patient in situ.

Preferably, a proximal end of the guide means, being proximal to a patient in use, comprises a generally cone-shaped tip.

Preferably, the proximal end of the guide means is blunted.

Preferably, the proximal end of the guide means is generally frusto-conical.

Preferably, the proximal end of the guide means has a smooth outer surface.

Advantageously, the proximal end of the guide means is shaped and textured such that it causes no damage and minimal discomfort to the patient during insertion and when located in situ.

Preferably, a proximal end of the guide means, being proximal to a patient in use, is weighted so it tends to drop down.

Advantageously, the proximal end of the guide means helps maintain the body in the desired position in the patient's pharynx and guide the device into the patient when reclined in use.

Preferably, a distal end of the guide means, being distal to a patient in use, comprises an anchor.

Advantageously, the distal end of the guide means is adapted to remain external to the patient's nasal cavity, and to be affixed to an external object (E.g. the patient's facial anatomy to help secure the body in the desired position in use).

Preferably, the anchor is one or more wings that contact an outer surface of at least one of the nares in use.

Preferably, the anchor is a clip adapted for clipping onto at least one of the nares in use. Preferably, the anchor comprises a face mask, nasal mask, head band or head gear.

Preferably, the anchor is adapted to engage the septum.

Advantageously, the anchor is adapted to comfortably fix the guide means to minimize movement of the guide means within the nares, nasal cavity and nasopharynx, thereby minimising discomfort and damage to the patient.

Preferably, a cross-section of a main length of the guide means fits within a diameter of 1 mm.

Preferably, a cross-section of a main length of the guide means fits within a diameter of 2 mm.

Advantageously, the guide means is sufficiently narrow such that it is comfortable for the patient and facilitates minimal interference with the breathing of the patient in use, yet facilitates easy guidance of the body into the desired position in the pharynx.

Inflation/Deflation

Preferably, the guide means defines a fluid passage.

Preferably, the fluid passage is an inflation passage and has an internal cross-sectional area of between 1 and 3 mm².

Preferably, the inflation passage has an internal cross-sectional area of between 1.5 and 2 mm².

Advantageously, the inflation passage has an internal cross-sectional area that is small enough such that it is comfortable for the patient and facilitates minimal interference with the breathing of the patient in use. Nevertheless, the internal cross-sectional area is sufficiently large such that a fluid can be communicated through the inflation passage.

Preferably, the guide means comprises a lumen, the lumen defining the fluid passage.

Advantageously, a lumen is one option for the fluid passage.

Preferably, the body comprises an internal volume that is fluid inflatable such that when inflated with fluid the body is in the expanded configuration and when deflated the body is in the collapsed configuration.

Advantageously, the body is easily configurable between the expanded and collapsed configurations by inflating and deflating the internal volume respectively.

Preferably, the fluid passage is in fluid communication with the internal volume.

Advantageously, the fluid passage provides a passage through which easy inflation and deflation of the body is facilitated.

Preferably, a distal end of the fluid passage, being distal to a patient in use, comprises a port adapted for connection to a fluid supply.

Advantageously, the distal end of the fluid passage can be connected to a fluid supply to supply fluid to inflate the body.

Preferably, the fluid supply is a pressurized fluid supply.

Advantageously, the fluid supply is adapted to spontaneously supply fluid to inflate the body once it is engaged with port.

Preferably, the body comprises an outer surface that is adapted to slowly release an anesthetic substance.

Advantageously, a local anesthetic is released to the tissues of the airway with which the body comes into contact, to minimize discomfort and soreness to the patient, and to prevent the stimulation of a potential gag reflex in use.

Preferably, the outer surface is adapted to be impregnated with the anesthetic substance.

Advantageously, impregnation of the anesthetic substance into the outer surface of the body is an effective way to facilitate slow release of the anesthetic substance when the outer surface of the body comes into contact with the tissues of the patient's airway.

Preferably, the body comprises a plurality of micropores extending from the internal volume to outside the body.

Preferably, the body comprises a plurality of nanopores extending from the internal volume to outside the body.

Advantageously, micropores and/or nanopores can be used to duct and slowly release of the anesthetic substance.

Preferably, the fluid is a gas.

Preferably, the fluid is air.

Advantageously, air supplies are readily available and cost-effective.

Preferably, the fluid comprises an anesthetic gas.

Advantageously, the anesthetic gas can be conducted to the surface of the body to provide anesthetic relief to the tissues of the patient with which the body comes into contact.

Preferably, the fluid is a liquid.

Preferably, the fluid is water.

Advantageously, water supplies are cost-effective and readily available. Preferably, the fluid comprises an anesthetic liquid.

Advantageously, the anesthetic liquid can be conducted to the surface of the body to provide anesthetic relief to the tissues of the patient with which the body comes into contact.

Materials

Preferably, the body and the guide means are made of a biocompatible material.

Advantageously, the body and guide means are made of material(s) that elicit little or no immune response from the patient in use.

Preferably, the guide means is made of plastic.

Preferably, the guide means is made from a plastic out of the group of plastics comprising: polyvinyl chloride, polyethylene, PEEK, Ultem PEI, Polysulfone, polypropylene and polyurethane.

Advantageously, the guide means is flexible, pliable, semi-rigid, and/or bendable into different shapes.

Preferably, the guide means is made of silicone.

Preferably, the body is made of plastic.

Preferably, the body is made from a plastic out of the group of plastics comprising: polyvinyl chloride, polyethylene, PEEK, Ultem PEI, Polysulfone, polypropylene and polyurethane.

Preferably, the body is made of silicone.

Advantageously, the body is made of a material that is sufficiently elastic such that it is inflatable and is comfortable for the patient in use.

Preferably, the body comprises a foam portion that is adapted to be compressed when the body is in the collapsed configuration.

Advantageously, when the body is expanded to the expanded configuration, the foam portion becomes uncompressed and helps resist the collapse of the patient's airway in use.

Twin Body Device

Preferably, the device for maintaining an oropharyngeal airway further comprises a further body adapted for configuration between a collapsed and an expanded configuration,
wherein in the expanded configuration, the further body is adapted for location in the nasopharynx or upper oropharynx, to define a pharyngeal airway and to maintain the pharyngeal airway at least sufficiently open for breathing when the further body is subjected to an additional external pressure,
the pharyngeal airway being substantially larger when the further body is in the expanded configuration than when the further body is in the collapsed configuration,
the further body being connected to the guide means,
the guide means being adapted to direct the further body through one of the nares and nasal cavity,
the further body comprising a further internal volume that is fluid inflatable such that when inflated with fluid the further body is in the expanded configuration and when deflated the further body is in the collapsed configuration.

Advantageously, the further body prevents obstruction of the patient's airway caused by the collapse of the airway in the nasopharyngeal region or in the upper oropharyngeal region, which may occur separately, or in addition to, collapse of the patient's natural airway in the oropharyngeal region adjacent to the base of the tongue.

Advantageously, the device comprising both the body and the further body is suited for use in a patient in whom obstruction at more than one region of the airway is anticipated.

Preferably, the fluid passage is in fluid communication with the further internal volume of the further body.

Advantageously, the body and further body are adapted to be inflated through the same fluid passage and by the same fluid supply.

Preferably, the additional external pressure is applied by the posterior region of the soft palate.

Advantageously, the further body is effective in resisting the collapse of the soft palate. As apnea and hypopnea during sleep, anesthesia or sedation can be caused by the collapse of the soft palate into the nasopharynx and/or upper oropharynx causing obstruction of the airway, the device is therefore effective in maintaining the pharyngeal airway at least sufficiently open in use so that the patient is able to continue breathing.

Preferably, the additional external pressure is applied by the nasopharyngeal wall.

Advantageously, the further body is effective in resisting the collapse of the nasopharyngeal wall. As with apnea and hypopnea during sleep, anesthesia or sedation can be caused by the collapse of the nasopharyngeal wall into the nasopharynx and/or upper oropharynx causing obstruction of the airway, the device is therefore effective in maintaining the pharyngeal airway at least sufficiently open in use so that the patient is able to continue breathing.

Preferably, the additional external pressure is applied by the oropharyngeal wall.

Advantageously, the further body is effective in resisting the collapse of the oropharyngeal wall. As with apnea and hypopnea during sleep, anesthesia or sedation can be caused by the collapse of the oropharyngeal wall into the nasopharynx and/or upper oropharynx causing obstruction of the airway, the device is therefore effective in maintaining the pharyngeal airway at least sufficiently open in use so that the patient is able to continue breathing.

Preferably, in the expanded configuration, the further body is adapted to apply a pressure to one or more areas from the following group of areas:
the nasopharyngeal wall;
the oropharyngeal wall;
the posterior pharyngeal wall; and
the posterior region of the soft palate.

Advantageously, the further body is effective in resisting the collapse of one or a combination of the nasopharyngeal wall, the oropharyngeal wall, the posterior pharyngeal wall, and the posterior region of the soft palate. As apnea and hypopnea during sleep, anesthesia or sedation can be caused by the collapse of one or a combination of the abovementioned tissues into the oropharynx, causing obstruction of the airway, the device is therefore effective in maintaining the pharyngeal airway at least sufficiently open in use so that the patient is able to continue breathing.

Twin Body Device by Location

Preferably, the further body is adapted for location at a location upstream of the body in use.

Preferably, at the location in use, the further body is not in contact with the body.

Advantageously, the further body does not interfere with the function of the body in situ.

Definition of Twin Body Device by Size & Shape

Preferably, the further body has a length of between 10 and 40 mm.

Preferably, the further body has a length of between 20 and 35 mm.

Preferably, the further body has a length of between 25 and 30 mm.

Preferably, the further body has a maximum width of between 4 and 12 mm.

Preferably, the further body has a maximum width of between 5 and 10 mm.

Advantageously, the further body in the expanded configuration is adapted to comfortably and snugly fit into the nasopharyngeal region or upper oropharyngeal region of the patient's airway, and to provide sufficient pressure against the collapsible regions of the patient's airway to maintain the airway at least sufficiently open for breathing.

Preferably, in the collapsed configuration, the further body fits within a diameter of 10 mm.

Preferably, in the collapsed configuration, the further body fits within a diameter of 7 mm.

Preferably, in the collapsed configuration, the further body fits within a diameter of about 6 mm.

Advantageously, minimizing the size of the further body in the collapsed configuration improves the ease of maneuverability of the further body into the patient's natural airway, thus minimizing the discomfort experienced by the patient during the insertion procedure. Once located in the desired position within the patient's natural airway, the further body can then be deployed into the expanded configuration to define the oropharyngeal airway.

Preferably, the pharyngeal airway has a minimum cross-sectional area of between 4 and 9 mm² when the further body is in the expanded configuration.

Preferably, the pharyngeal airway has a minimum cross-sectional area of between 6 and 8 mm² when the further body is in the expanded configuration.

Advantageously, the pharyngeal airway is sufficiently large in use to facilitate breathing with minimal resistance. This will allow the patient to breathe naturally and comfortably, which, if the device is used for treatment of OSA, encourages compliance.

Advantageously, the pharyngeal airway is sufficiently large in use to enable it to be used for PAP during anesthesia and sedation.

Preferably, in the expanded configuration, the further body is generally torus shaped.

Advantageously, a torus shape is a shape that defines an internal passageway, the passageway forming the pharyngeal airway.

Preferably, in the expanded configuration, the further body has an external diameter than is smaller than the external diameter of the body in the expanded configuration.

Advantageously, as the nasopharyngeal region or upper oropharyngeal region is generally smaller than the middle to lower oropharyngeal region, the further body is adapted to be located in nasopharyngeal region or upper oropharyngeal region, while the body is adapted to be located in the middle to lower oropharyngeal region.

Preferably, in the expanded configuration, the further body has an external diameter of between 4 mm and 15 mm.

Preferably, the external diameter is between 5 mm and 12 mm.

Advantageously, the further body in the expanded configuration is adapted to comfortably and snugly fit into the nasopharyngeal and/or upper oropharyngeal region of a patient's airway, and to provide sufficient pressure against the collapsible tissues of the patient's airway to maintain the airway at least sufficiently open for breathing.

Preferably, in the expanded configuration, the further body has an internal diameter of between 3 mm and 12 mm.

Preferably, the internal diameter is between 3 mm and 7 mm.

Preferably, the further body is a generally thin-walled structure.

Advantageously, the further body is proportioned such that the cross-sectional area of the pharyngeal airway is maximized to facilitate breathing with minimal resistance in use. Nevertheless, the further body is adapted to sufficiently withstand the application of the additional external pressure from the collapsible tissues of the patient's airway in use.

Preferably, the device for maintaining an oropharyngeal airway comprises one or more fold lines to control movement of the further body between the expanded and the collapsed configuration.

Advantageously, the further body is easily and reliably configurable between the collapsed and expanded configuration in use.

Preferably, when the further body is in the collapsed configuration it is twisted.

Preferably, when the further body is in the expanded configuration, the pharyngeal airway comprises an opening towards the nasopharynx and another opening towards the mouth and tongue.

Advantageously, the further body is adapted to maintain the oropharyngeal airway open in a region of the oropharynx proximal to the nasopharynx of the patient, and in a region of the oropharynx proximal to the mouth of the patient, facilitating both breathing through the nose and through the mouth, respectively.

Preferably, the further body is sized to be received within a pediatric patient.

Preferably, the further body is sized to be received within an adult patient.

Advantageously, the device can be produced in various sizes to fit both adults and children.

Inflation/Deflation of Twin Body Device

Preferably, the further body comprises an outer surface that is adapted to slowly release an anesthetic substance.

Advantageously, a local anesthetic is released to the tissues of the airway with which the further body comes into contact, to minimize discomfort and soreness to the patient, and to prevent the stimulation of a potential gag reflex in use.

Preferably, the outer surface is adapted to be impregnated with the anesthetic substance.

Advantageously, impregnation of the anesthetic substance into the outer surface of the further body is an effective way to facilitate slow release of the anesthetic substance when the outer surface of the further body comes into contact with the tissues of the patient's airway.

Preferably, the further body comprises a plurality of micropores extending from the internal volume to outside the further body.

Preferably, the further body comprises a plurality of nanopores extending from the internal volume to outside the further body.

Advantageously, micropores and/or nanopores can be used to duct and slowly release of the anesthetic substance.

Materials

Preferably, the further body is made of a biocompatible material.

Advantageously, the further body is made of material(s) that elicit little or no immune response from the patient in use.

Preferably, the further body is made of plastic.

Preferably, the further body is made of a plastic out of the group of plastics comprising: polyvinyl chloride, polyethylene, PEEK, Ultem PEI, Polysulfone, polypropylene and polyurethane.

Preferably, the further body is made of silicone.

Advantageously, the further body is made of a material that is sufficiently elastic such that it is inflatable and is comfortable for the patient in use.

Preferably, the further body comprises a foam portion that is adapted to be compressed when the further body is in the collapsed configuration.

Advantageously, when the further body is expanded to the expanded configuration, the foam portion becomes uncompressed and helps resist the collapse of the patient's airway in use.

Other Variations

Preferably, in the expanded configuration, an outer surface of the body comprises one or more grooves adapted to allow fluids to drain when the outer surface is in contact with an anatomical surface in use.

Preferably, in the expanded configuration, an outer surface of the further body comprises one or more grooves adapted to allow fluids to drain when the outer surface is in contact with an anatomical surface in use.

Advantageously, the grooves prevent the buildup of fluids produced by the patient in the pharynx in use.

Preferably, an outer surface of at least a portion of the guide means is adapted to slowly release an anesthetic substance in use.

Advantageously, a local anesthetic is released to the tissues of the nares, nasal cavity and naxopharynx with which the guide means comes into contact, to minimize discomfort and soreness and prevent the stimulation of a potential gag reflex in use.

Preferably, the outer surface is adapted to be impregnated with the anesthetic substance.

Advantageously, impregnation of the anesthetic substance into the outer surface of the guide means is an effective way to facilitate slow release of the anesthetic substance.

Preferably, the body is adapted to move between the expanded and collapsed configuration on application of an electric current.

Preferably, the body comprises one or more electro-active polymer elements and the body is adapted to move between the expanded and collapsed configuration on application of an electric current to the one or more electro-active polymer elements.

Advantageously, the body is configurable between the expanded and collapsed configurations by application of an electric current.

Preferably, the guide means comprises a wire adapted for connection to a selective power source at one end and to the one or more electro-active polymer elements at the other end.

Advantageously, this allows the application of an electric current to be safely and effectively performed.

Preferably, the further body is adapted to move between the expanded and collapsed configuration on application of an electric current.

Preferably, the further body comprises one or more electro-active polymer elements and the further body is adapted to move between the expanded and collapsed configuration on application of an electric current to the one or more electro-active polymer elements.

Advantageously, the body is configurable between the expanded and collapsed configurations by application of an electric current.

Preferably, the guide means comprises a wire adapted for connection to a selective power source at one end and to the one or more electro-active polymer elements at the other end.

Advantageously, this allows the application of an electric current to be safely and effectively performed.

Preferably, in the expanded configuration, the body is generally cylindrical in shape.

Advantageously, a cylindrical shape is a shape that defines an internal diameter, and hence is capable of defining the oropharyngeal airway.

Preferably, the body comprises an inner membrane, an outer membrane and a membrane volume in between, the outer membrane comprising pore means, the body being such that when in the expanded configuration, any gel or fluid in the membrane volume is dispensed through the pore means to outside the body.

Advantageously, an anesthetic, analgesic, medication or lubricant may be stored within the membrane volume, in the form of a gel or fluid, and may be delivered via the pore means to outside the body, and thus to the anatomical surface of the patient that is in contact with the outer surface of the body, in use.

Preferably, in the expanded configuration, the further body is generally cylindrical in shape.

Advantageously, a cylindrical shape is a shape that defines an internal diameter, and hence is capable of defining the pharyngeal airway.

Preferably, the further body comprises an inner membrane, an outer membrane and a membrane volume in between, the outer membrane comprising pore means, the further body being such that when in the expanded configuration, any gel or fluid in the membrane volume is dispensed through the pore means to outside the further body.

Advantageously, an anesthetic, analgesic, medication or lubricant may be stored within the membrane volume, in the form of a gel or fluid, and may be delivered via the pore means to outside the further body, and thus to the anatomical surface of the patient that is in contact with the outer surface of the further body, in use.

Nasal Laryngeal Mask Airway

According to a second aspect of the present invention, a device for maintaining an oropharyngeal airway is provided, comprising:
a body adapted for configuration between a collapsed and an expanded configuration,
wherein in the expanded configuration, the body is adapted for location in the oropharynx, to define an oropharyngeal airway and to maintain the oropharyngeal airway at least sufficiently open for breathing when the body is subjected to an external pressure,
the oropharyngeal airway being substantially larger when the body is in the expanded configuration than when the body is in the collapsed configuration.

Advantageously, the device for maintaining an oropharyngeal airway can be used in a surgical context for securing the patency of the patient's airway perioperatively, while the patient is anaesthetised or sedated, or postoperatively.

Pressure

Preferably, the external pressure is applied by the base of the tongue.

Advantageously, the device for maintaining an oropharyngeal airway is effective in resisting the collapse of the base of the tongue. As with apnea and hypopnea during sleep, anesthesia or sedation can be caused by the collapse of the base of the tongue into the oropharynx causing obstruction of the airway, the device is therefore effective in maintaining the oropharyngeal airway at least sufficiently open in use so that the patient is able to continue breathing.

Preferably, the external pressure is applied by the oropharyngeal wall.

Advantageously, the device for maintaining an oropharyngeal airway is effective in resisting the collapse of the oropharyngeal wall. As with apnea and hypopnea during sleep, anesthesia or sedation can be caused by the collapse of the oropharyngeal wall into the oropharynx causing obstruction of the airway, the device is therefore effective in maintaining the oropharyngeal airway at least sufficiently open in use so that the patient is able to continue breathing.

Preferably, in the expanded configuration, the body is adapted to apply a pressure to one or more areas from the following group of areas:
the oropharyngeal wall;
the posterior pharyngeal wall;
the base of the tongue;
the soft palate; and
one or more supraglottic structures.

Advantageously, the device for maintaining an oropharyngeal airway is effective in resisting the collapse of one or a combination of the oropharyngeal wall, the posterior pharyngeal wall, the base of the tongue, the soft palate, and other supraglottic structures. As with apnea and hypopnea during sleep, anesthesia or sedation can be caused by the collapse of one or a combination of the abovementioned tissues into the oropharynx, causing obstruction of the airway, the device is therefore effective in maintaining the oropharyngeal airway at least sufficiently open in use so that the patient is able to continue breathing.

Location

Preferably, in the expanded configuration, the body is adapted for location around at least a portion of the glottis, epiglottis and laryngeal opening.

Preferably, in the expanded configuration, the body is adapted for location around the glottis, epiglottis and laryngeal opening.

Advantageously, when the body is located in the desired position in use and expanded into the expanded configuration, it is substantially fixed in place by its location around the glottis, epiglottis and laryngeal opening.

Advantageously, when the body is located in the desired position in use and expanded into the expanded configuration, the body provides support for the supraglottic structures by encircling the glottis with the anterior portion in the vallecula and the posterior portion in the oesophageal entrance.

Size & Shape

Preferably, the body has a length of between 10 and 40 mm.

Preferably, the body has a length of between 20 and 35 mm.

Preferably, the body has a length of between 25 and 30 mm.

Preferably, in the expanded configuration, the body is generally boot-shaped.

Preferably, in the expanded configuration, at the heel of the boot, the body has a maximum width of between 10 and 30 mm.

Preferably, in the expanded configuration, at the heel of the boot, the body has a maximum width of between 15 and 25 mm.

Preferably, in the expanded configuration, the body is generally kidney-shaped.

Advantageously, the body in the expanded configuration is adapted to comfortably and snugly fit into the region of a patient's airway at which the glottis, epiglottis and laryngeal opening is located, and to provide sufficient pressure against collapse of the tissues of the patient's airway to maintain the airway at least sufficiently open for breathing.

Preferably, the oropharyngeal airway has a minimum cross-sectional area of between 19 and 60 mm² when the body is in the expanded configuration.

Preferably, the oropharyngeal airway has a minimum cross-sectional area of between 20 and 50 mm² when the body is in the expanded configuration.

Preferably, in the expanded configuration, at the heel of the boot, the body has an external diameter of between 15 and 30 mm.

Preferably, at the heel of the boot, the body has an external diameter of between 15 and 25 mm.

Preferably, at the heel of the boot, in the expanded configuration, the body has an internal diameter of between 5 and 12 mm.

Preferably, at the heel of the boot, the body has an internal diameter of between 5 and 9 mm.

Preferably, the body is a generally thin-walled structure.

Advantageously, the body is proportioned such that, when in the expanded configuration, the cross-sectional area of the oropharyngeal airway is maximized to facilitate breathing with minimal resistance in use. Nevertheless, the body is adapted to sufficiently withstand the application of the external pressure from the collapsible tissues of the patient's airway in use.

Preferably, the device for maintaining an oropharyngeal airway comprises one or more fold lines to control movement of the body between the expanded and the collapsed configuration.

Advantageously, the body is easily and reliably configurable between the collapsed and expanded configuration in use.

Preferably, when the body is in the collapsed configuration it is twisted.

Preferably, when the body is in the expanded configuration, the oropharyngeal airway comprises an opening towards the nasopharynx and another opening towards the mouth and tongue.

Advantageously, the body facilitates both breathing through the nose and through the mouth.

Preferably, the body is sized to be received within a pediatric patient.

Preferably, the body is sized to be received within an adult patient.

Advantageously, the device can be produced in various sizes to fit both adults and children.

Naso-Pharyngeal Airway Means

Preferably, the device for maintaining an oropharyngeal airway further comprises nasopharyngeal airway means connected to the body and defining a nasopharyngeal airway, the nasopharyngeal airway being in fluid communication with the oropharyngeal airway.

Preferably, the nasopharyngeal airway means is adapted for configuration between a collapsed and an expanded configuration, such that in the collapsed configuration, it can be readily passed through one of the nares, nasal cavity and nasopharynx and into the pharynx, and in the expanded configuration a user can breathe through the nasopharyngeal airway.

Advantageously, in use, the patient ventilates through the nasopharyngeal airway means and the oropharyngeal airway.

Preferably, the nasopharyngeal airway means comprises a thermo-sensitive material such that at least one material property of the thermo-sensitive material changes as a function of temperature.

Preferably, the thermo-sensitive material changes shape as a function of temperature such that the nasopharyngeal airway means changes shape as a function of temperature.

Advantageously, the shape of the nasopharyngeal airway means is be adapted to change shape after insertion such that it better conforms to the natural contours of the nares, nasal cavity and nasopharynx of the patient for greater comfort in use.

Preferably, the nasopharyngeal airway means has an external diameter of between 6 and 12 mm.

Preferably, the nasopharyngeal airway means has an external diameter of between 7 and 10 mm.

Advantageously, the nasopharyngeal airway means is sufficiently narrow such that it is relatively comfortable for the patient in use.

Preferably, the nasopharyngeal airway has a minimum internal diameter of between 5 and 10 mm.

Preferably, the nasopharyngeal airway has a minimum internal diameter of between 6 and 7 mm.

Preferably, the nasopharyngeal airway has a minimum cross-sectional area of between 15 and 80 mm².

Preferably, the nasopharyngeal airway has a minimum cross-sectional area of between 25 and 50 mm².

Advantageously, the nasopharyngeal airway means facilitates breathing with less resistance.

Preferably, the nasopharyngeal airway means has a wall thickness of between 0.1 and 10 mm.

Preferably, the nasopharyngeal airway means has a wall thickness of between 0.3 and 0.5 mm.

Advantageously, the wall of the nasopharyngeal airway means provides for a significant internal cross-sectional area of the nasopharyngeal airway, while maintaining the nasopharyngeal airway clear, unobstructed and uncollapsed within the patient's nasal passage, nasopharynx and pharynx in use.

Inflation/Deflation

Preferably, the body comprises an internal volume that is fluid inflatable such that when inflated with fluid the body is in the expanded configuration and when deflated the body is in the collapsed configuration.

Advantageously, the body is easily configurable between the expanded and collapsed configurations by inflating and deflating the internal volume respectively.

Preferably, the nasopharyngeal airway means is adapted to extend from at least the nares through the nasal cavity and nasopharynx and into the pharynx.

Advantageously, the nasopharyngeal airway means provides a clear and unobstructed airway through which the patient is able to ventilate.

Preferably, the nasopharyngeal airway means comprises an inflation fluid passage having a fluid port adapted for connection to a fluid supply, the inflation fluid passage being in fluid communication with the internal volume.

Advantageously, the body can be readily inflated and deflated via the inflation fluid passage.

Preferably, when the inflation fluid passage is inflated at least a portion of the length of the nasopharyngeal airway means is in the expanded configuration and when deflated, the portion of the nasopharyngeal airway means is in the collapsed configuration.

Advantageously, the nasopharyngeal airway means is easily configurable between the expanded and the collapsed configurations by inflating and deflating the inflation fluid passage, respectively.

Advantageously, the body and the nasopharyngeal airway means both are adapted to be inflated through the inflation fluid passage and by the same fluid supply.

Preferably, the fluid port comprises an airway connector.

Advantageously, the airway connector is adapted for connection to an air supply for the inflation of the body.

Preferably, the body and nasopharyngeal airway means move to the expanded configuration when a fluid pressure is applied at the fluid port and to the collapsed configuration when the fluid is withdrawn through the fluid port.

Advantageously, the body and the inflation fluid passage need only to be inflated and deflated at the commencement and termination of use, respectively, and therefore does not require the application of constant fluid pressure to maintain the body and inflation fluid passage in the expanded configuration.

Preferably, the inflation fluid passage has an internal cross-sectional area of between 0.5 and 2 mm².

Preferably, the inflation fluid passage has an internal cross-sectional area of between 1 and 1.5 mm².

Advantageously, the inflation fluid passage is sufficiently large such that a fluid may be conducted within the inflation fluid passage with minimal resistance, yet has a sufficiently small internal cross-sectional area to allow the cross-sectional area of the nasopharyngeal airway to be sufficient within the nasopharyngeal airway means.

Preferably, the inflation fluid passage is a lumen.

Advantageously, a lumen is a way of defining the inflation fluid passage.

Preferably, the nasopharyngeal airway means is a dual, thin-walled structure, the inflation fluid passage provided by the volume between outer and inner walls of the dual, thin-walled structure.

Advantageously, this variation of the nasopharyngeal airway means, having the inflation fluid passage located between the inner and outer walls, minimizes interruption within the nasopharyngeal airway, thereby minimizing the resistance to breathing of the user in use.

Preferably, the inflation fluid passage is one or more inflation fluid sub-passages.

Advantageously, compromise of one sub-passage due to puncture or otherwise will not jeopardise the functionality of the entire device.

Preferably, the fluid supply is a pressurized fluid supply.

Advantageously, the fluid supply is adapted to spontaneously supply fluid to inflate the body once engaged.

Preferably, an outer surface of the nasopharyngeal airway means is adapted to slowly release an anesthetic substance.

Advantageously, release of a local anesthetic to the tissues of the nasal cavity, nasopharynx and pharynx with which the nasopharyngeal airway means comes into contact minimizes discomfort and soreness to the patient and reduces the potential stimulation of a potential gag reflex in use.

Preferably, the outer surface is adapted to be impregnated with the anesthetic substance.

Advantageously, impregnation of the anesthetic substance into the outer surface of the nasopharyngeal airway means is an effective way to facilitate slow release of the anesthetic substance when the outer surface of the nasopharyngeal airway means comes into contact with the tissues of the patient's airway.

Preferably, the nasopharyngeal airway means comprises a plurality of micropores extending from the inflation fluid passage to outside the nasopharyngeal airway means.

Preferably, the nasopharyngeal airway means comprises a plurality of nanopores extending from the inflation fluid passage to outside the nasopharyngeal airway means.

Advantageously, micropores and/or nanopores can be used to duct and slowly release the anesthetic substance.

Preferably, an outer surface of the body is adapted to slowly release an anesthetic substance.

Advantageously, release of a local anesthetic to the tissues of the nasal cavity, nasopharynx and pharynx with which the body comes into contact minimizes discomfort and soreness to the patient and reduces the potential stimulation of a potential gag reflex in use.

Preferably, the outer surface is adapted to be impregnated with the anesthetic substance.

Advantageously, impregnation of the anesthetic substance into the outer surface of the body is an effective way to facilitate slow release of the anesthetic substance when the outer surface of the body comes into contact with the tissues of the patient's airway.

Preferably, the body comprises a plurality of micropores extending from the internal volume to outside the body.

Preferably, the body comprises a plurality of nanopores extending from the internal volume to outside the body.

Advantageously, micropores and/or nanopores can be used to duct and slowly release the anesthetic substance.

Preferably, the fluid is a gas.

Preferably, the fluid is air.

Advantageously, air supplies are cost-effective and readily available.

Preferably, the fluid comprises an anesthetic gas.

Advantageously, the anesthetic gas can be conducted to the surface of the body to provide anesthetic relief to the tissues of the patient with which the body comes into contact.

Preferably, the fluid is a liquid.

Preferably, the fluid is water.

Advantageously, water is cost-effective and readily available.

Preferably, the fluid comprises an anesthetic liquid.

Advantageously, the anesthetic liquid can be conducted to the surface of the body to provide anesthetic relief to the tissues of the patient with which the body comes into contact.

Guide Means

Preferably, the device for maintaining an oropharyngeal airway further comprises guide means connected to the body.

Preferably, the guide means is a nasopharyngeal guide means adapted to direct the body in the collapsed configuration through one of the nares, nasal cavity and nasopharynx and into the pharynx.

Advantageously, the body is easily and readily inserted and directed into the desired position in the patient's pharynx. If correctly inserted, the patient may feel minimal discomfort during and following the insertion procedure, and will not likely sustain any damage to the nares, nasal cavity, nasopharynx or pharynx.

Advantageously, insertion of the device and the location of the device in situ is relatively comfortable and well-tolerated, allowing the device to be inserted while the patient is awake. This allows the patient's airway to be secured prior to the induction of anesthesia or sedation, reducing the likelihood of difficult intubation, and of anesthesia-related mortality and morbidity caused by loss of the patency of the airway.

Advantageously, the patient's airway can be secured during maxillofacial or oral procedures with perfect oral access.

Preferably, the guide means is adapted to direct the body such that it does not apply a substantial pressure to the region of the turbinate at the entrance to the nasopharynx.

Preferably, the guide means is adapted to direct the body such that it does not contact the region of the turbinate at the entrance to the nasopharynx.

Advantageously, as the region of the turbinate is a sensitive region, the body can be inserted and directed into place without the patient experiencing excessive discomfort or suffering damage in the region of the turbinate.

Preferably, the guide means is flexible.

Preferably, the guide means is semi-rigid.

Preferably, the guide means is pliable.

Preferably, the length of the guide means is bendable into different shapes.

Advantageously, the guide means is adapted to facilitate ease of insertion of the body into the pharynx.

Preferably, the guide means comprises a thermo-sensitive material such that at least one material property of the thermo-sensitive material changes as a function of temperature.

Preferably, the thermo-sensitive material changes shape as a function of temperature such that the guide means changes shape as a function of temperature.

Advantageously, the shape of the guide means is be adapted to change shape after insertion such that it better conforms to the natural contours of the nares, nasal cavity and nasopharynx of the patient for greater comfort in use.

Preferably, the guide means comprises a shape memory material and is bendable into a new shape when heated and retains the new shape when cooled.

Advantageously, the guide means is be adapted to be tailored to shape to better conform to the natural contours of the nares, nasal cavity and nasopharynx of each individual patient, making it more comfortable for the patient in use.

Preferably, the guide means comprises a cord.

Preferably, the guide means has an external cross-sectional area of between 1.5 and 4 mm².

Preferably, the guide means has an external cross-sectional area of between 2 and 3.5 mm².

Advantageously, the guide means is sufficiently narrow such that it is comfortable for the patient and facilitates minimal interference with the breathing of the patient in use, yet facilitates easy guidance of the body into the desired position in the pharynx.

Preferably, the guide means comprises one or more bend regions, each bend region being adapted to move between a first angle and a second angle.

Advantageously, mechanical manipulation of the guide means is limited to a pre-defined range of movement, which prevents the guide means from being bent out of shape or damaged.

Preferably, at least one of the bend regions is adapted to move between the first angle and the second angle upon application of an electric current.

Preferably, the at least one bend region comprises an electro-active shape memory polymer element and is adapted to move between the first angle and the second angle upon application of an electric current to the electro-active shape memory polymer element.

Preferably, the electro-active shape memory polymer element is a piezo-electric polymer element.

Preferably, at least one of the bend regions is adapted to move between the first angle and the second angle upon application of light within a predetermined wavelength range.

Preferably, the at least one bend region comprises a light-induced shape memory polymer element and is adapted to move between the first angle and the second angle upon application of a light within a predetermined wavelength range to the light-induced shape memory polymer element.

Preferably, the at least one bend region comprises an electromechanical servo motor and is adapted to move between the first angle and the second angle upon application of an electric current to the electromechanical servo motor.

Preferably, the guide means comprises a series of elements connected by hinge means, and a tensioning means, the guide means being adapted to move between a first configuration in which the series of elements are disposed relative to each other in a first disposition and a second configuration in which the series of elements are disposed relative to each other in a second disposition by tensioning of the tensioning means.

Preferably, the series of elements are hollow and the tensioning means is a tensioning cord that extends through the series of elements.

Preferably, the guide means comprises a series of elements connected by hinge means, and a push means, the guide means being adapted to move between a first configuration in which the series of elements are disposed relative to each other in a first disposition and a second configuration in which the series of elements are disposed relative to each other in a second disposition by pushing of the push means.

Preferably, the series of elements are hollow and the push means extends through the series of elements.

Preferably, the guide means comprises a sheath means and a rotational member located therein, the rotational member being connected to the body at one end and extending externally from the sheath means at the other end, such that when the rotational member is rotated relative to the sheath means in a first direction the body twists to the collapsed configuration and when the rotational member is rotated relative to the sheath means in the other direction the body un-twists to the expanded configuration.

Preferably, the guide means comprises a sheath means and a push-pull member located therein, the push-pull member being connected to the body at one end and extending externally from the sheath means at the other end, such that when the push-pull member is moved relative to the sheath means in one direction the body collapses to the collapsed configuration and in the other direction the body expands to the expanded configuration.

Advantageously, the guide means can be adapted to be mechanically manipulated by any one or more of a range of different means, to dispose it in a first configuration to optimize it for ease of insertion, and in a second configuration to optimize it for comfort to the patient in situ.

Preferably, a proximal end of the guide means, being proximal to a patient in use, comprises a generally cone-shaped tip.

Preferably, the proximal end of the guide means is blunted.

Preferably, the proximal end of the guide means is generally frusto-conical. Preferably, the proximal end of the guide means has a smooth outer surface.

Advantageously, the proximal end of the guide means is shaped and textured such that it causes minimal or no damage or discomfort to the patient during insertion and when located in situ.

Preferably, a proximal end of the guide means, being proximal to a patient in use, is weighted so it tends to drop down.

Advantageously, the proximal end of the guide means helps maintain the body in the desired position and orientation and guide the device into the patent in use.

Preferably, a distal end of the guide means, being distal to a patient in use, comprises an anchor.

Advantageously, the distal end of the guide means is adapted to remain external to the patient's nasal cavity and to be affixed to an external object (E.g. the patient's facial anatomy to help secure the body in the desired position in use).

Preferably, the anchor is one or more wings that contact an outer surface of at least one of the nares in use.

Preferably, the anchor is a clip adapted for clipping onto at least one of the nares in use. Preferably, the anchor comprises a face mask, nasal mask, head band or head gear.

Preferably, the anchor is adapted to engage the septum.

Advantageously, the anchor is adapted to comfortably fix the guide means to minimize movement of the guide means within the nares, nasal cavity and nasopharynx, thereby minimising discomfort and damage to the patient.

Preferably, a cross-section of a main length of the guide means fits within a diameter of 2.5 mm.

Preferably, a cross-section of a main length of the guide means fits within a diameter of 2 mm.

Advantageously, the guide means is sufficiently narrow such that it is comfortable for the patient and facilitates minimal interference with the breathing of the patient in use, yet facilitates easy guidance of the body into the desired position in the pharynx.

Preferably, the nasopharyngeal airway means comprises a nasopharyngeal guide means adapted to direct the body in the collapsed configuration through one of the nares, nasal cavity and nasopharynx and into the pharynx.

Advantageously, the body is easily and readily inserted and directed into the desired position, such that the patient feels less discomfort, and does not sustain damage to the nares, nasal cavity, nasopharynx or pharynx.

Materials

Preferably, the body, the nasopharyngeal airway means and the guide means are made of a biocompatible material.

Advantageously, the body and guide means are made of material(s) that elicit little or no immune response from the patient in use.

Preferably, the guide means is made of plastic.

Preferably, the guide means is made from a plastic out of the group of plastics comprising: polyvinyl chloride, polyethylene, PEEK, Ultem PEI, Polysulfone, polypropylene and polyurethane.

Advantageously, the guide means is flexible, pliable, semi-rigid, and/or bendable into different shapes.

Preferably, the guide means is made of silicone.

Preferably, the body is made of plastic.

Preferably, the body is made from a plastic out of the group of plastics comprising: polyvinyl chloride, polyethylene, PEEK, Ultem PEI, Polysulfone, polypropylene and polyurethane.

Preferably, the body is made of silicone.

Advantageously, the body is made of a material that is sufficiently elastic such that it is inflatable and is comfortable for the patient in use.

Preferably, the nasopharyngeal airway means is made of plastic.

Preferably, the nasopharyngeal airway means is made from a plastic out of the group of plastics comprising: polyvinyl chloride, polyethylene, PEEK, Ultem PEI, Polysulfone, polypropylene and polyurethane.

Preferably, the nasopharyngeal airway means is made of silicone.

Advantageously, the nasopharyngeal airway means is made of a material that is sufficiently elastic such that it is inflatable and is comfortable for the patient in use.

Preferably, the body comprises a foam portion that is adapted to be compressed when the body is in the collapsed configuration.

Advantageously, when the body is expanded to the expanded configuration, the foam portion becomes uncompressed and helps resist the collapse of the patient's airway in use.

Other Features

Preferably, in the expanded configuration, an outer surface of the body comprises one or more grooves adapted to allow fluids to drain when the outer surface is in contact with an anatomical surface in use.

Advantageously, the grooves prevent the buildup of fluids produced by the patient in the pharynx in use.

Preferably, an outer surface of at least a portion of the guide means is adapted to slowly release an anesthetic substance in use.

Advantageously, a local anesthetic is released to the tissues of the nares, nasal cavity and naxopharynx with which the guide means comes into contact, to reduce discomfort and soreness and prevent the stimulation of a potential gag reflex in use.

Preferably, the outer surface is adapted to be impregnated with the anesthetic substance.

Advantageously, impregnation of the anesthetic substance into the outer surface of the guide means is an effective way to facilitate slow release of the anesthetic substance.

Preferably, in the expanded configuration, an outer surface of the nasopharyngeal airway means comprises one or more grooves adapted to allow fluids to drain when the outer surface is in contact with an anatomical surface in use.

Advantageously, the grooves prevent the buildup of fluids produced by the patient in the nasal passage and nasopharynx in use.

Preferably, the body is adapted to move between the expanded and collapsed configuration on application of an electric current.

Preferably, the body comprises one or more electro-active polymer elements and is adapted to move between the expanded and collapsed configuration on application of an electric current to the one or more electro-active polymer elements.

Preferably, the nasopharyngeal airway means comprises a wire adapted for connection to a power source at one end and to the one or more electro-active polymer elements at the other end.

Advantageously, the body and/or nasopharyngeal airway means are configurable between the expanded and collapsed configurations by application of an electric current.

Preferably, the body comprises an inner membrane, an outer membrane and a membrane volume in between, the outer membrane comprising pore means, the body being such that when in the expanded configuration, any gel or fluid in the membrane volume is dispensed through the pore means to outside the body.

Advantageously, an anesthetic, analgesic, medication or lubricant may be stored within the membrane volume, in the form of a gel or fluid, and may be delivered via the pore means to outside the body, and thus to the anatomical surface of the patient that is in contact with the outer surface of the body in use.

Preferably, the device for maintaining an oropharyngeal airway further comprises oesophageal fluid canal means defining an oesophageal fluid canal.

Preferably, a proximal end of the oesophageal fluid canal comprises an opening towards the esophagus and a distal end is located outside of the nares of the patient in use.

Advantageously, the oesphageal fluid canal is adapted to conduct emesis substances from the esophagus, from the proximal end to the distal end of the canal, thereby reducing aspiration of the emesis substances into the patient's airway in use.

Oropharyngeal Cushion—Method Claim

According to a third aspect of the present invention, a method of creating an oropharyngeal airway is provided, comprising the steps of:
inserting a guide means through one of the nares, nasal cavity, supraglottic region, and nasopharynx and into the pharynx to locate an expandable body in a collapsed configuration in the pharynx, adjacent the base of the tongue above the cartilaginous epiglottis; and
expanding the expandable body from the collapsed configuration to an expanded configuration to create the oropharyngeal airway through the expandable body.

Advantageously, the expandable body is adapted to prevent the patient's pharynx from collapsing in the region of the oropharynx that is adjacent the base of the tongue, and to create an oropharyngeal airway through which the patient is able to ventilate.

Advantageously, the method of creating an oropharyngeal airway can be used in the treatment of obstructive sleep apnea.

Advantageously, the method of creating an oropharyngeal airway can be used in a surgical context for securing the patency of the patient's airway perioperatively, while the patient is anaesthetized or sedated, or postoperatively.

Advantageously, the method of creating an oropharyngeal airway allows the expandable body to be easily manoeuvred into the patient's airway in the collapsed configuration, and once located in the desired position within the patient's airway, to be deployed into the expanded configuration.

Advantageously, the method of creating an oropharyngeal airway allows the expandable body to be easily and readily inserted and directed into the desired position by aid of the guide means, such that the patient feels reduced discomfort, and does not sustain damage to the nares, nasal cavity, supraglottic region, nasopharynx or pharynx.

Advantageously, the method of creating an oropharyngeal airway allows the intake of air into the patient's airways to be naturally humidified and filtered by the patient's own nasal passages and/or oral passage in use.

Advantageously, the method of creating an oropharyngeal airway can be easily learnt and readily employed by a person who is not a medical practitioner, including the patient himself or herself.

Preferably, the guide means comprises an inflation passage, the body being in fluid communication with the inflation passage and inflatable from the collapsed configuration to the expanded configuration, and the step of expanding the expandable body comprises the step of:
delivering a fluid through the inflation passage to the body to inflate the body from the collapsed configuration to the expanded configuration.

Advantageously, the method of creating an oropharyngeal airway allows for the body to be easily configurable between the expanded and collapsed configurations by inflating and deflating the body respectively.

Preferably, the fluid is a gas.

Preferably, the fluid is air.

Advantageously, air supplies are readily available and cost-effective.

Preferably, the fluid comprises an anesthetic gas.

Advantageously, the anesthetic gas can be used to effectively inflate the body and provide local anesthetic to reduce discomfort to the patient.

Preferably, the fluid is a liquid.

Preferably, the fluid is water.

Advantageously, water supplies are readily available and cost-effective.

Preferably, the fluid comprises an anesthetic liquid.

Advantageously, the anesthetic liquid can be used to effectively inflate the body and provide local anesthetic to reduce discomfort to the patient.

Nasal Laryngeal Mask Airway—Method Claim

According to a fourth aspect of the present invention, a method of creating an oropharyngeal airway is provided, comprising the steps of:
inserting a nasopharyngeal airway means in a collapsed configuration through one of the nares, nasal cavity and nasopharynx and into the pharynx to locate an expandable body in a collapsed configuration in the pharynx substantially around the glottis, epiglottis and laryngeal opening; and
expanding the nasopharyngeal airway means and the expandable body from the collapsed configuration to an expanded configuration to create the oropharyngeal airway through the nasopharyngeal airway means and expandable body.

Advantageously, the expandable body is adapted to prevent the patient's pharynx from collapsing, and to create an oropharyngeal airway and nasopharyngeal airway through which the patient is able to ventilate.

Advantageously, the method of creating an oropharyngeal airway can be used in a surgical context, for securing the patency of the patient's airway perioperatively, while the patient is anaesthetised or sedated, or postoperatively.

Advantageously, the method of creating an oropharyngeal airway allows the expandable body to be manoeuvred into the patient's airway in the collapsed configuration such that the patient feels reduced discomfort, and does not sustain damage to the nares, nasal cavity, nasopharynx or pharynx. Once located in the desired position within the patient's airway, the expandable body is adapted to be deployed into the expanded configuration.

Advantageously, the method is readily performed on an awake patient, and thus the patient's airway is advantageously secured prior to the induction of sedation or anesthesia, reducing the likelihood of difficult intubation, and of anesthesia-related mortality and morbidity caused by loss of the patency of the airway.

Preferably, the nasopharyngeal airway means comprises an inflation passage and is inflatable from the collapsed configuration to the expanded configuration, the body is inflatable from the collapsed configuration to the expanded configuration and is in fluid communication with the inflation passage, and the step of expanding the nasopharyngeal airway means and the expandable body comprises the step of:
delivering a fluid into the inflation passage and thus also into the body to inflate the nasopharyngeal airway means and the body from the collapsed configuration to the expanded configuration.

Advantageously, the method of creating an oropharyngeal airway allows for the nasopharyngeal airway means and the expandable body to be easily configurable between the expanded and collapsed configurations by inflating and deflating the nasopharyngeal airway means and the expandable body, respectively.

Preferably, the fluid is a gas.

Preferably, the fluid is air.

Advantageously, air supplies are readily available and cost-effective.

Preferably, the fluid comprises an anesthetic gas.

Advantageously, the anesthetic gas can be used to effectively inflate the body and provide local anesthetic to minimize discomfort to the patient.

Preferably, the fluid is a liquid.

Preferably, the fluid is water.

Advantageously, water supplies are readily available and cost-effective.

Preferably, the fluid comprises an anesthetic liquid.

Advantageously, the anesthetic liquid can be used to effectively inflate the body and provide local anesthetic to minimize discomfort to the patient.

System Claim

According to a fifth aspect of the present invention, a system for maintaining an oropharyngeal airway is provided, comprising:
a device for maintaining an oropharyngeal airway as described above, further comprising a fluid port, the fluid port being in fluid communication with the internal volume; and an inflation means connected to the fluid port and adapted to deliver a fluid into the internal volume of the body.

Preferably, the inflation means is a piston-cylinder arrangement.

Advantageously, the inflation means facilitates fast and efficient inflation of the body to the expanded configuration.

Preferably, the piston-cylinder arrangement is a syringe.

Advantageously, the inflation means is portable, easy to use, economically produced, and does not require connection to an electrical power source.

Preferably, the inflation means is an electrically powered air pump.

Advantageously, the inflation means facilitates fast and efficient inflation of the body to the expanded configuration.

Preferably, the inflation means is a finger operated air pump.

Advantageously, the inflation means is portable, easy to use, economically produced, and does not require connection to an electrical power source.

Preferably, the inflation means is further adapted to withdraw the fluid from the internal volume of the body.

Advantageously, withdrawing the fluid from the internal volume at the close of the treatment is able to be performed using the same means as for delivery of the same fluid.

Preferably, the fluid is a gas.

Preferably, the fluid is air.

Advantageously, air supplies are readily available and cost-effective.

Preferably, the fluid comprises an anesthetic gas.

Advantageously, the anesthetic gas can be used to effectively inflate the body and provide local anesthetic to minimize discomfort to the patient.

Preferably, the fluid is a liquid.

Preferably, the fluid is water.

Advantageously, water supplies are readily available and cost-effective.

Preferably, the fluid comprises an anesthetic liquid.

Advantageously, the anesthetic liquid can be used to effectively inflate the body and provide local anesthetic to reduce discomfort to the patient.

Preferably, the system for maintaining an oropharyngeal airway further comprises an overpressurisation valve adapted to open and release pressure from the internal volume when the pressure is higher than a predefined pressure limit.

Advantageously, the system is adapted to detect when the internal volume is overpressurised and to spontaneously remedy the overpressurisation if and when it is detected, thereby allowing the device to be used with a high level of safety in the patient.

Other aspects of the invention are also disclosed.

### BRIEF DESCRIPTION OF THE DRAWINGS

Notwithstanding any other forms which may fall within the scope of the present invention, first and second preferred embodiments of the invention will now be described, by way of example only, with reference to the accompanying drawings in which:

FIG. 1 shows a left side view of a device for maintaining an oropharyngeal airway in a collapsed configuration located in situ in a patient's pharynx, shown in partial cutaway, in accordance with a preferred embodiment of the present invention;

FIG. 2 shows a left side view of the device for maintaining an oropharyngeal airway of FIG. 1 in an expanded configuration located in situ in a patient's pharynx, shown in partial cutaway; and

FIG. 3 shows (i) a left side view of a device for maintaining an oropharyngeal airway in an expanded configuration located in situ in a patient's pharynx, shown in partial cutaway, and (ii) a cross-sectional view of a body of the device taken along line A-A, in accordance with another preferred embodiment of the present invention.

### DETAILED DESCRIPTION OF SPECIFIC EMBODIMENTS

It should be noted in the following description that like or the same reference numerals in different embodiments denote the same or similar features. In the following description, all uses of ‘proximal’ and ‘distal’ as terms of reference are defined to mean proximal and distal to a patient in use, respectively.

Oropharyngeal Cushion

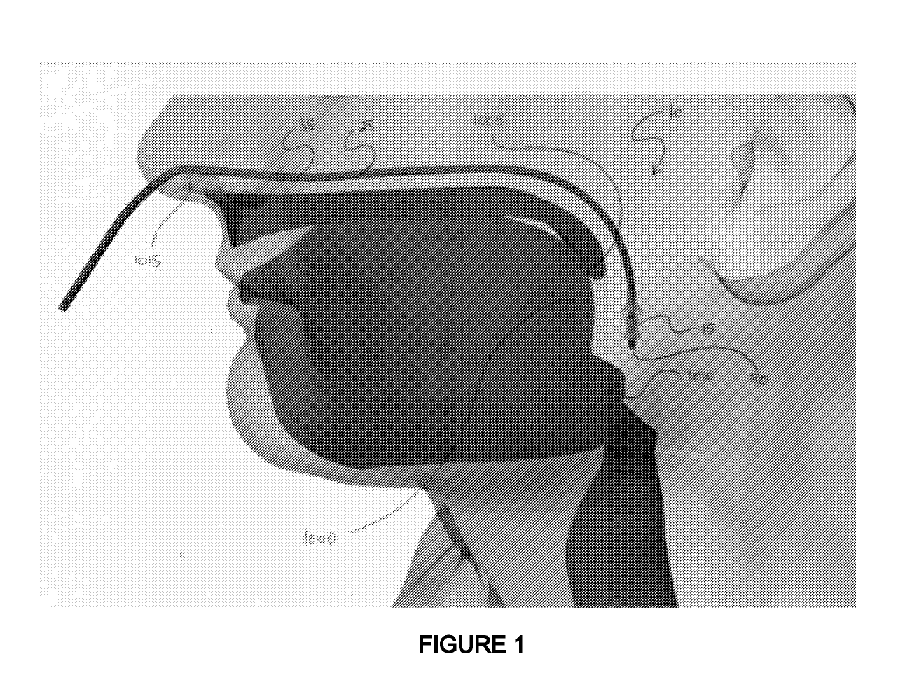

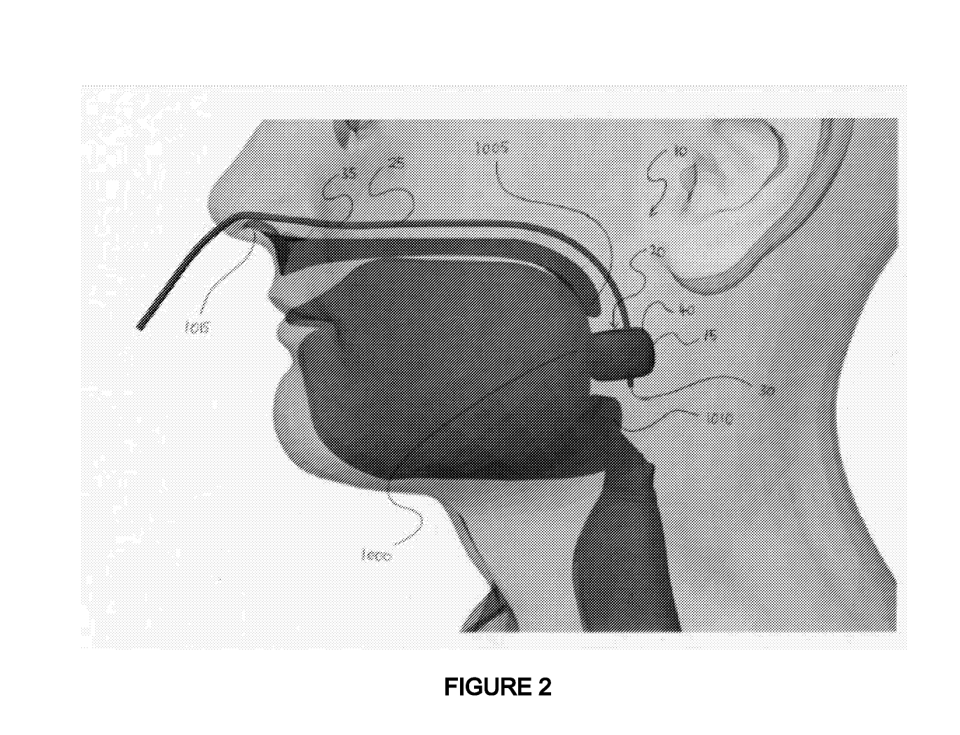

Referring to FIGS. 1 and 2, a device 10 for maintaining an oropharyngeal airway at least sufficiently open for breathing is shown. The device 10 comprises a body 15 adapted for configuration between a collapsed configuration as shown in FIG. 1, and an expanded configuration, as shown in FIG. 2. The body 15, in the expanded configuration (see FIG. 2) is adapted for location in the oropharynx, to define an oropharyngeal airway 20, such that when the device 10 is fitted in a patient, and the body 15 is in the expanded configuration, the patient is able to breathe through the oropharyngeal airway 20 through his or her own respiratory effort. The body 15 in the expanded configuration is adapted to maintain the oropharyngeal airway 20 at least sufficiently open for breathing despite being subjected to an external pressure. The oropharyngeal airway 20 is substantially larger when the body 15 is in the expanded configuration than when the body 15 is in the collapsed configuration. Indeed, in this embodiment, the oropharyngeal airway 20 is undefined when the body 15 is in the collapsed configuration (see FIG. 1).

The device 10 for maintaining the oropharyngeal airway 20 can be used for the treatment of obstructive sleep apnea. The device 10 for maintaining the oropharyngeal airway 20 can also be used in a surgical context, for securing the patency of the patient's airway perioperatively, while the patient is anaesthetised or sedated, or postoperatively.

Pressure

The body 15, in the expanded position is adapted to maintain the oropharyngeal airway 20 at least sufficiently open for breathing when the body 15 is subjected to external pressure applied by the base of the tongue 1000, the oropharyngeal wall, the posterior pharyngeal wall, the soft palate 1005, or any combination of the above.

Hypopnea and apnea during sleep, anesthesia or sedation, is often caused by the collapse of one or a combination of the above-mentioned tissues into the oropharynx causing obstruction of the natural airway. The device 10 for maintaining the oropharyngeal airway 20 is adapted to resist the collapse of one or a combination of the above-mentioned tissues in use, and maintain the oropharyngeal airway 20 at least sufficiently open in use so that the patient is able to continue breathing.

Location

The body 15 is adapted to be located in the oropharynx, at a position upstream of the cartilaginous epiglottis 1010. It is this region of the natural airway that may become obstructed and cause apnea or hypopnea. In the preferred embodiment, the body 15 is not in contact with the cartilaginous epiglottis 1010, as it is important that the body 15 in use does not interfere with the function of the epiglottis 1010, and that the body 15 is not displaced from its position within the oropharynx by movement of the epiglottis 1010.

Size & Shape

The length of the body 15 should be between 10 and 40 mm. In the preferred embodiment, the length of the body 15 is between 25 and 30 mm. The maximum width of the body 15 should be between 5 and 12 mm. In the preferred embodiment, the maximum width of the body 15 is between 6 and 10 mm. The body 15 is sized to allow the body 15 in the expanded configuration to comfortably and snugly fit into the oropharyngeal region of the patient's natural airway, and to provide sufficient pressure against the collapsible tissues of the patient's airway to maintain the airway at least sufficiently open for breathing. In practice, the body 15 is to be provided in various sizes to accommodate different patients. In one embodiment, the body 15 is sized to be received within a pediatric patient. In another embodiment, the body 15 is sized to be received within an adult patient.

The body 15, in the collapsed configuration, should fit within a diameter of about 10 mm. Referring to FIG. 1, in the preferred embodiment, the body 15 in the collapsed configuration fits within a diameter of about 6 mm. It is advantageous to minimize the size of the body 15 in the collapsed configuration, as a smaller body 15 is more easily maneuverable into the patient's natural airway, and thus the discomfort experienced by the patient during the insertion procedure is minimized. Once located in the desired position within the patient's natural airway, the body 15 can then be deployed into the expanded configuration to define the oropharyngeal airway 20.

The oropharyngeal airway 20 should have a minimum cross-sectional area of between 6 and 13 mm² when the body 15 is in the expanded configuration. In the preferred embodiment, the oropharyngeal airway 20 has a minimum cross-sectional area of between 7 and 9 mm² when the body 15 is in the expanded configuration. It is ideal that the oropharyngeal airway 20 is sufficiently large in use to facilitate breathing with minimal resistance. This will allow the patient to breathe naturally and comfortably, which, if the device 10 is used for treatment of OSA, encourages compliance.

Referring to FIG. 2, the body 15 in the expanded configuration is generally torus shaped, the torus shape defining an internal passageway that forms the oropharyngeal airway 20. In the expanded configuration, the body 15 should have an external diameter that is between 5 mm and 12 mm. In the preferred embodiment, the body 15 has an external diameter that is between 6 mm and 10 mm. The body 15 is sized to allow the body 15 in the expanded configuration to comfortably and snugly fit into the oropharyngeal region of the patient's natural airway, and to provide sufficient pressure against the collapsible tissues of the patient's airway to maintain the airway at least sufficiently open for breathing. In practice, the body 15 is to be provided in various sizes to accommodate different patients.

In the expanded configuration, the body 15 should have an internal diameter of between 4 mm and 9 mm. As the internal passageway of the body 15 defines the oropharyngeal airway 20, it is ideal that the internal diameter of the body 15 is sufficiently large to facilitate breathing through the oropharyngeal airway 20 with minimal resistance. In this arrangement, the internal passageway through the body 15 has a corresponding internal cross-sectional area of generally between 12.0 and 65 mm², thereby satisfying the minimum cross-sectional area requirements described above for the oropharyngeal airway 20 when the body 15 is in the expanded configuration.

In the preferred embodiment, in the expanded configuration, the body 15 has an internal diameter of between 4 mm and 8 mm. In this arrangement, the internal passageway defined by the further body has an internal cross-sectional area of generally between 12.0 and 55 mm².

The body 15 is a generally thin-walled structure, which allows the cross-sectional area of the oropharyngeal airway 20 to be maximized. Nevertheless, the body 15, in the expanded configuration, is adapted to sufficiently withstand the application of external pressure from the collapsible tissues of the patient's natural airway in use.

In one embodiment (not shown), the device for maintaining an oropharyngeal airway comprises one or more fold lines to control movement of the body between the expanded and the collapsed configuration. In another embodiment (not shown), when the body is in the collapsed configuration it is twisted.

In another embodiment (not shown), when the body is in the expanded configuration, the oropharyngeal airway comprises an opening towards the nasopharynx and another opening towards the mouth and tongue. Thus, the body in the expanded configuration is adapted to maintain the oropharyngeal airway open both in a region of the oropharynx proximal to the nasopharynx of the patient, and in a region of the oropharynx of the patient proximal to the mouth of the patient. This facilitates breathing both through the nose and through the mouth. Visually, the body is substantially Y-shaped, with a first of the two branches of the Y-shape opening towards the nasopharynx, a second of the two branches of the Y-shape opening towards the mouth and tongue, and the trunk of the Y-shape opening towards the trachea of the patient.

Guide Means

Referring to FIGS. 1 and 2, the device 10 for maintaining the oropharyngeal airway 20 further comprises guide means 25 connected to the body 15. The guide means 25 is a nasopharyngeal guide means adapted to direct the body 15 in the collapsed configuration through one of the nares 1015, nasal cavity and nasopharynx and into the pharynx. In this embodiment, the body 15 is easily and readily inserted and directed into the desired position in the patient's pharynx. If correctly inserted, the patient will feel minimal discomfort during and following the insertion procedure, and will not sustain any damage to the nares 1015, nasal cavity, nasopharynx or pharynx. Insofar as the device 10 for maintaining the oropharyngeal airway 20 is used for treating patients suffering from obstructive sleep apnea, the ease and comfort with which the body 15 can be inserted facilitates compliance by the patient in self-administration of the treatment. Moreover, the device 10 is adapted to be easily directed into place by a person who is not a medical practitioner, including by the patient himself or herself, and is thus suitable for regular home use.

It is a significant advantage of the device 10 that it allows the intake of air into the patient's airways to be naturally humidified and filtered by the patient's own nasal passages and/or oral passage in use, unlike other supraglottic devices used for airway management in the sedated or anaesthetised patient, which facilitates breathing substantially through a tube that bypasses the natural humidification and filtration of the patient's nasal passage and/or oral passage. Furthermore, unlike positive airway pressure (PAP) devices used to treat obstructive sleep apnea, air pressure through the patient's airway is not required, and thus some of the associated adverse effects of PAP, such as nasal or oral dryness, are substantially eliminated.

As previously described, insertion of the device 10 and the maintenance of the device 10 in situ are relatively comfortable and well-tolerated, allowing the device 10 to be inserted in a tolerant patient while the patient is awake. Significantly, this enables the patient's airway to be secured prior to the induction of anesthesia or sedation, reducing the likelihood of difficult intubation, and of anesthesia-related mortality and morbidity caused by loss of the patency of the airway. The device 10 can be especially advantageous for use in maxillofacial or oral procedures, as the device 10 is adapted to access the patient's natural airway through the nasal passages, allowing unobstructed access to the patient's mouth to be gained.

When used correctly, the guide means 25 is adapted to direct the body 15 such that it does not contact the region of the turbinate at the entrance to the nasopharynx, or at least does not apply substantial pressure to the region of the turbinate at the entrance of the nasopharynx, during and following insertion of the device 10. The body 15 can thus be inserted and directed into place without the patient experiencing excessive discomfort or suffering damage in the region of the turbinate, which is a very sensitive region.

In the preferred embodiment, the guide means 25 is connected to a region of the body 15 that is posterior to the oropharyngeal airway 20 in use when the body 15 is in the expanded configuration (see FIG. 2). The location of the guide means 25 thus ensures that it does not interfere with, narrow, or in any way restrict the oropharyngeal airway 20 in use.

In the preferred embodiment, the guide means 25 is flexible and pliable, but sufficiently semi-rigid, such that it facilitates ease of insertion of the body 15 into the pharynx. In one embodiment, the length of the guide means 25 is bendable into different shapes.

In another embodiment, the guide means 25 comprises a thermo-sensitive material such that at least one material property of the thermo-sensitive material, such as shape, changes as a function of temperature. In one embodiment, following insertion, the guide means 25 is adapted to absorb heat from the patient's tissues. Once sufficiently warm, the guide means 25 is adapted to change into a shape that better conforms to the natural contours of the nares 1015, nasal cavity and nasopharynx of the patient, making it more comfortable for the patient in use.

In another embodiment, the guide means 25 comprises a shape memory material such that it is bendable into a new shape when heated and retains the new shape when cooled. An advantage of this embodiment is that it allows the guide means 25 to be tailored to shape to better conform to the natural contours of the nares 1015, nasal cavity and nasopharynx of each individual patient, making it more comfortable for the patient in use.

In another embodiment, the guide means comprises a cord. The cord is adapted to be long enough to be located external to the patient's nares 1015 in use such that retraction of the device is readily achieved when desired.

The guide means 25 should have an external cross-sectional area of between 0.5 and 3 mm². In the preferred embodiment, the guide means 25 has an external cross-sectional area of between 1 and 2 mm². The guide means 25 is thus sufficiently narrow such that it is comfortable for the patient and facilitates minimal interference with the breathing of the patient in use, yet facilitates easy guidance of the body 15 into the desired position in the patient's pharynx.

In one embodiment, the guide means comprises one or more bend regions, each bend region being adapted to move between a first angle and a second angle. This allows mechanical manipulation of the guide means to be limited to a pre-defined range of movement, which prevents the guide means from being bent out of shape or damaged.

In another embodiment, at least one of the bend regions is adapted to move between the first angle and the second angle upon application of an electric current. In another embodiment, the at least one bend region comprises an electro-active shape memory polymer element and is adapted to move between the first angle and the second angle upon application of an electric current to the electro-active shape memory polymer element. In one embodiment, the electro-active shape memory polymer element is a piezo-electric polymer element.

In another embodiment, at least one of the bend regions is adapted to move between the first angle and the second angle upon application of light within a predetermined wavelength range. In another embodiment, the at least one bend region comprises a light-induced shape memory polymer element and is adapted to move between the first angle and the second angle upon application of a light within a predetermined wavelength range to the light-induced shape memory polymer element.

In another embodiment, the at least one bend region comprises an electromechanical servo motor and is adapted to move between the first angle and the second angle upon application of an electric current to the electromechanical servo motor.

In another embodiment, the guide means comprises a series of elements connected by hinge means, and a tensioning means, the guide means being adapted to move between a first configuration in which the series of elements are disposed relative to each other in a first disposition and a second configuration in which the series of elements are disposed relative to each other in a second disposition by tensioning of the tensioning means. In one embodiment, the series of elements are hollow and the tensioning means is a tensioning cord that extends through the series of elements.

In another embodiment, the guide means comprises a series of elements connected by hinge means, and a push means, the guide means being adapted to move between a first configuration in which the series of elements are disposed relative to each other in a first disposition and a second configuration in which the series of elements are disposed relative to each other in a second disposition by pushing of the push means. In another embodiment, the series of elements are hollow and the push means extends through the series of elements.

In another embodiment, the guide means comprises a sheath means and a rotational member located therein, the rotational member being connected to the body at one end and extending externally from the sheath means at the other end, such that when the rotational member is rotated relative to the sheath means in a first direction the body twists to the collapsed configuration and when the rotational member is rotated relative to the sheath means in the other direction the body un-twists to the expanded configuration.

In another embodiment, the guide means comprises a sheath means and a push-pull member located therein, the push-pull member being connected to the body at one end and extending externally from the sheath means at the other end, such that when the push-pull member is moved relative to the sheath means in one direction the body collapses to the collapsed configuration and in the other direction the body expands to the expanded configuration.

The guide means 25 can be adapted to be mechanically manipulated by any one or more of a range of different means, to dispose it in a first configuration to optimize it for ease of insertion, and in a second configuration to optimize it for comfort to the patient in situ.

Referring to FIGS. 1 and 2, a proximal end 30 of the guide means 25 comprises a generally cone-shaped tip. In this embodiment, the proximal end 30 of the guide means 25 can be more specifically described as a blunted and frusto-conical tip. The texture of outer surface of the proximal end 30 of the guide means 25 is smooth. Thus, the proximal end 30 is adapted to cause no damage and minimal discomfort to the patient during insertion and when located in situ. Furthermore, the proximal end 30 of the guide means 25 is weighted so that it tends to drop down, helping to maintain the body 15 in the desired position and orientation within the patient's pharynx in use.

In one embodiment (not shown), a distal end of the guide means comprises an anchor. Thus, the distal end of the guide means is adapted to remain external to the patient's nasal cavity, and to be affixed to an external region of the patient's facial anatomy to help secure the body in the desired position in use. In another embodiment, the guide means is adapted to be of a pre-determined length and to terminate in the anchor. The anchor, once attached to a part of the patient's facial anatomy, limits any further insertion of the guide means than is desired, and as previously described, helps maintain the body in the desired position in use. Thus, correct positioning of the body into the patient's pharynx is easily ascertainable. The length of the guide means can be tailored to suit each individual patient.

In one embodiment, the anchor is one or more wings that contact an outer surface of at least one of the nares 1015 in use. In another embodiment, the anchor is a clip adapted for clipping onto at least one of the nares in use. In another embodiment, the anchor comprises a face mask, nasal mask, head band or head gear. In another embodiment, the anchor is adapted to engage the septum. In any one of the embodiments of the anchor, the anchor is adapted to comfortably fix the guide means to minimize movement of the guide means within the nares 1015, nasal cavity and nasopharynx, thereby minimising discomfort to the patient and/or damage to the patient's tissues in use.

A cross-section of a main length of the guide means 25 should fit within a diameter of 1 mm. Referring to FIGS. 1 and 2, in the preferred embodiment, the cross-section of the main length of the guide means 25 fits within a diameter of 2 mm. The guide means 25 is sufficiently narrow such that it is comfortable for the patient and facilitates minimal interference with the breathing of the patient in use, yet facilitates easy guidance of the body 15 into the desired position in the pharynx.

Inflation/Deflation

Referring to FIGS. 1 and 2, the guide means 25 is a substantially a hollow tubular structure that defines a lumen. The lumen defines a fluid passage 35, which will hereinafter be referred to as an inflation passage 35. The inflation passage 35 should have an internal cross-sectional area of between 1 and 3 mm². In the preferred embodiment, the inflation passage 35 has an internal cross-sectional area of between 1.5 and 2 mm². Thus, the inflation passage 35 has an internal cross-sectional area that is sufficiently small such that it is comfortable for the patient and facilitates minimal interference with the breathing of the patient in use. Nevertheless, the internal cross-sectional area is sufficiently large such that a fluid may be conducted within the inflation passage 35 with minimal resistance.

The body 15 comprises an internal volume 40 that is fluid inflatable such that when inflated with fluid the body 15 is in the expanded configuration (see FIG. 2) and when deflated the body 15 is in the collapsed configuration (see FIG. 1). The body 15 is thus easily configurable between the expanded and collapsed configurations by inflating and deflating the internal volume 40 respectively. The inflation passage 35 is in fluid communication with the internal volume 40, such that the internal volume 40 can be readily inflated or deflated via the inflation passage 35.

In one embodiment, a distal end of the inflation passage (not shown) comprises a port adapted for connection to a fluid supply. In the preferred embodiment, the port is a standard 22 mm airway connector. Thus, the distal end of the inflation passage can be connected to a fluid supply (not shown) to supply fluid to inflate the body. In one embodiment, the fluid supply is a pressurised fluid supply, enabling spontaneous inflation of the internal volume once the distal end of the inflation passage is engaged with the fluid supply.

In one embodiment, the body 15 comprises an outer surface that is adapted to slowly release an anesthetic substance. Release of a local anesthetic to the tissues of the airway with which the body 15 comes into contact minimizes discomfort and soreness to the patient, and prevents the stimulation of a potential gag reflex in use. In one embodiment, the outer surface of the body 15 is impregnated with the anesthetic substance prior to insertion, facilitating slow release of the anesthetic substance to the tissues of the patient's airway with which the outer surface of the body 15 comes into contact. In another embodiment, the body 15 comprises a plurality of micropores and/or nanopores extending from the internal volume 40 to outside the body 15. The micropores and/or nanopores can be adapted to duct and slowly release of the anesthetic substance to the tissues of the patient's airway with which the body 15 comes into contact.

In one embodiment, the inflation passage 35 is adapted to conduct a gas, such as air. Air supplies are readily available and cost-effective. In another embodiment, the inflation passage 35 is adapted to conduct a liquid, such as water.

In another embodiment, the inflation passage 35 is adapted to conduct an anesthetic gas, or an anesthetic liquid. The presence of micropores and/or nanopores in the surface of the body 15 in this embodiment would enable the anesthetic substance to be conducted to the surface of the body 15 to provide anesthetic relief to the tissues of the patient with which the body 15 comes into contact.

Materials

In the preferred embodiment, the body 15 and the guide means 25 are made of a biocompatible material, such that the device 10, in use, elicits little or no immune response from the patient.

In one embodiment, the guide means 25 is made of plastic. More specifically, the guide means 25 is made from a plastic out of the group of plastics comprising polyvinyl chloride, polyethylene, PEEK, Ultem PEI, Polysulfone, polypropylene and polyurethane. As described above, the guide means 25 is adapted to be flexible, pliable, semi-rigid, and/or bendable into different shapes. In another embodiment, the guide means is made of silicone.

In one embodiment, the body is made of plastic. More specifically, the body is made from a plastic out of the group of plastics comprising polyvinyl chloride, polyethylene, PEEK, Ultem PEI, Polysulfone, polypropylene and polyurethane. In another embodiment, the body 15 is made of silicone, which allows the body 15 to be sufficiently elastic such that it is inflatable and is comfortable for the patient in use.

In one embodiment, the body comprises a foam portion that is adapted to be compressed when the body is in the collapsed configuration. When the body is expanded to the expanded configuration, the foam portion becomes uncompressed and helps resist the collapse of the patient's airway in use.

Twin Body Device

In another embodiment (not shown), the device for maintaining an oropharyngeal airway further comprises a further body adapted for configuration between a collapsed and an expanded configuration. In the expanded configuration, the further body is adapted for location in the nasopharynx or upper oropharynx, to define a pharyngeal airway and to maintain the pharyngeal airway at least sufficiently open for breathing when the further body is subjected to an additional external pressure.

The pharyngeal airway is substantially larger when the further body is in the expanded configuration than when the further body is in the collapsed configuration, in a similar fashion to the body 15, which defines the oropharyngeal airway 20 to be substantially larger when the body 15 is in the expanded configuration than when the body 15 is in the collapsed configuration.

The further body is also connected to the guide means 25, such that the guide means 25 is adapted to direct the further body through one of the nares 1015 and nasal cavity, and into position in the nasopharynx or upper oropharynx of the patient. In this way, the body 15 and the further body are both directed into position using a single insertion procedure.

The further body comprises a further internal volume that is fluid inflatable such that when inflated with fluid the further body is in the expanded configuration and when deflated the further body is in the collapsed configuration, in a similar fashion to the internal volume 40 of the body 15.

The further body prevents obstruction of the patient's airway caused by the collapse of the patient's natural airway in the nasopharyngeal region or in the upper oropharyngeal region, which may occur separately, or in addition to, collapse of the patient's natural airway in the oropharyngeal region adjacent to the base of the tongue. Thus, in this embodiment, the device comprising both the body 15 and the further body is suited for use in a patient in whom obstruction at more than one region of the airway is anticipated. In the preferred embodiment, the further body in the expanded configuration is adapted to apply a pressure to the nasopharyngeal wall, the oropharyngeal wall, the posterior pharyngeal wall and/or the posterior region of the soft palate.

In this embodiment, the inflation passage 35 is in fluid communication with both the internal volume of the body 15 and the further inflation volume of the further body. In this way, the body 15 and further body are adapted to be inflated through the same inflation passage and by the same fluid supply.

The further body is adapted to maintain the pharyngeal airway at least sufficiently open for breathing when the additional external pressure is applied by the posterior region of the soft palate, the nasopharyngeal wall, the oropharyngeal wall, the posterior pharyngeal wall, or any combination of the above.

Hypopnea and apnea during sleep, anesthesia or sedation, is often caused by the collapse of one or a combination of the above-mentioned tissues into the nasopharynx and/or upper oropharynx causing obstruction of the natural airway. The device for maintaining an oropharyngeal airway is adapted to resist the collapse of one or a combination of the above-mentioned tissues in use, and maintain the pharyngeal airway at least sufficiently open in use so that the patient is able to continue breathing.

Twin Body Device By Location

The further body is adapted to be located at a location upstream of the body 15 in use, such that the further body is not in contact with the body 15. Thus, the further body does not interfere with the function of the body 15 in situ.

Definition of Twin Body Device by Size & Shape

The further body should have a length of between 10 and 40 mm. In the preferred embodiment, the further body has a length of between 25 and 30 mm. The further body should have a maximum width of between 4 and 12 mm. In the preferred embodiment, the further body has a maximum width of between 5 and 10 mm. The further body is sized to allow the further body in the expanded configuration to comfortably and snugly fit into the nasopharyngeal region or upper oropharyngeal region of a patient's natural airway, and to provide sufficient pressure against the collapsible regions of the patient's airway to maintain the airway at least sufficiently open for breathing. Like the body 15, the further body in practice is to be provided in various sizes to accommodate different patients. In one embodiment, the further body is sized to be received within a pediatric patient. In another embodiment, the further body is sized to be received within an adult patient.

In the collapsed configuration, the further body should fit within a diameter of 10 mm. In the preferred embodiment, the further body fits within a diameter of about 6 mm. It is advantageous to minimize the size of the further body in the collapsed configuration, as a smaller further body is more easily maneuverable into the patient's natural airway, and thus the discomfort experienced by the patient during the insertion procedure is minimized. Once located in the desired position within the patient's airway, the further body can then be deployed into the expanded configuration to define the pharyngeal airway.

The pharyngeal airway should have a minimum cross-sectional area of between 4 and 9 mm² when the further body is in the expanded configuration. In the preferred embodiment, the pharyngeal airway has a minimum cross-sectional area of between 6 and 8 mm² when the further body is in the expanded configuration. It is ideal that the pharyngeal airway is sufficiently large in use to facilitate breathing with minimal resistance. This will allow the patient to breathe naturally and comfortably, which, if the device is used for treatment of OSA, encourages compliance. Like the body 15 in the expanded configuration, the further body in the expanded configuration is generally torus shaped, the torus shape defining an internal passageway that forms the pharyngeal airway. In the expanded configuration, the further body has an external diameter than is smaller than the external diameter of the body 15 in the expanded configuration, as the nasopharyngeal region or upper oropharyngeal region is generally smaller than the middle to lower oropharyngeal region.

In the expanded configuration, the further body should have an external diameter of between 4 mm and 15 mm. In the preferred embodiment, the further body has an external diameter of between 5 mm and 12 mm. As described above, the further body is sized to allow the further body in the expanded configuration to comfortably and snugly fit into the nasopharyngeal or upper oropharyngeal region of the patient's airway, and to provide sufficient pressure against the collapsible tissues of the patient's airway to maintain the airway at least sufficiently open for breathing.

In the expanded configuration, the generally torus-shaped further body should have an internal diameter of between 3 mm and 12 mm. As the internal passageway of the further body defines the pharyngeal airway, it is ideal that the internal diameter of the further body is sufficiently large to facilitate breathing through the pharyngeal airway with minimal resistance.

In this arrangement, the internal passageway through the further body has a corresponding internal cross-sectional area of generally between 7.0 and 120 mm², thereby satisfying the minimum cross-sectional area requirements described above for the pharyngeal airway when the further body is in the expanded configuration.

In the preferred embodiment, in the expanded configuration, the generally torus-shaped further body has an internal diameter of between 3 mm and 7 mm. In this arrangement, the internal passageway defined by the further body has an internal cross-sectional area of generally between 7.0 and 40 mm².

The further body is a generally thin-walled structure, which allows the cross-sectional area of the pharyngeal airway to be maximized. Nevertheless, the further body in the expanded configuration is adapted to sufficiently withstand the application of the additional external pressure from the relevant collapsible tissues of the patient's airway in use.

In one embodiment, the further body comprises one or more fold lines to control movement of the further body between the expanded and the collapsed configuration. In another embodiment, when the further body is in the collapsed configuration it is twisted.

In another embodiment, when the further body is in the expanded configuration, the pharyngeal airway comprises an opening towards the nasopharynx and another opening towards the mouth and tongue. Thus, the further body in the expanded configuration is adapted to maintain the oropharyngeal airway open both in a region of the oropharynx proximal to the nasopharynx of the patient, and in a region of the oropharynx proximal to the mouth of the patient. This facilitates both breathing through the nose and through the mouth, respectively.

Inflation/Deflation of Twin Body Device

In one embodiment, the further body comprises an outer surface that is adapted to slowly release an anesthetic substance. Release of a local anesthetic to the tissues of the airway with which the further body comes into contact, minimizes discomfort and soreness to the patient, and prevents the stimulation of a potential gag reflex in use. In one embodiment, the outer surface of the further body is impregnated with the anesthetic substance prior to insertion, facilitating slow release of the anesthetic substance to the tissues of the airway with which the outer surface of the further body comes into contact. In another embodiment, the further body comprises a plurality of micropores and/or nanopores extending from the internal volume to outside the further body, the micropores and/or nanopores being adapted to duct and slowly release the anesthetic substance to the tissues of the patient's airway with which the further body comes into contact.

Materials of Twin Body Device

The further body is made of a biocompatible material, such that it elicits little or no immune response from the patient in use.

In one embodiment, the further body is made of plastic. More specifically, the further body is made from a plastic out of the group of plastics comprising polyvinyl chloride, polyethylene, PEEK, Ultem PEI, Polysulfone, polypropylene and polyurethane. In another embodiment, the further body is made of silicone, which allows the further body to be sufficiently elastic such that it is inflatable and is comfortable for the patient in use.

In one embodiment, the further body comprises a foam portion that is adapted to be compressed when the further body is in the collapsed configuration. When the further body is expanded to the expanded configuration, the foam portion becomes uncompressed and helps resist the collapse of the patient's airway in use.

Other Variations

In one embodiment, in the expanded configuration, an outer surface of the body comprises one or more grooves adapted to allow fluids to drain when the outer surface is in contact with an anatomical surface in use. In another embodiment, in the expanded configuration, an outer surface of the further body comprises one or more grooves adapted to allow fluids to drain when the outer surface is in contact with an anatomical surface in use. The grooves prevent the buildup of fluids produced by the patient in the pharynx in use.

In one embodiment, an outer surface of at least a portion of the guide means is adapted to slowly release an anesthetic substance in use, to minimize discomfort and soreness and prevent the stimulation of a potential gag reflex in use. In one embodiment, the outer surface of the guide means is adapted to be impregnated with the anesthetic substance, which is an effective way to facilitate slow release of the anesthetic substance.

In another embodiment, the body is adapted to move between the expanded and collapsed configuration on application of an electric current. In another embodiment, the body comprises one or more electro-active polymer elements and the body is adapted to move between the expanded and collapsed configuration on application of an electric current to the one or more electro-active polymer elements. In one embodiment, the guide means comprises a wire adapted for connection to a selective power source at one end and to the one or more electro-active polymer elements at the other end, allowing the application of an electric current to be safely and effectively performed.

In another embodiment, further body is adapted to move between the expanded and collapsed configuration on application of an electric current. In another embodiment, the further body comprises one or more electro-active polymer elements and the further body is adapted to move between the expanded and collapsed configuration on application of an electric current to the one or more electro-active polymer elements. In another embodiment, the guide means comprises a wire adapted for connection to a selective power source at one end and to the one or more electro-active polymer elements at the other end, allowing the application of an electric current to be safely and effectively performed.

In another embodiment, the body in the expanded configuration is generally cylindrical in shape, comprising an internal passageway that defines the oropharyngeal airway.

In another embodiment, the body comprises an inner membrane, an outer membrane and a membrane volume in between, the outer membrane comprising pore means, the body being such that when in the expanded configuration, any gel or fluid in the membrane volume is dispensed through the pore means to outside the body. This allows an anesthetic, analgesic, medication or lubricant to be stored within the membrane volume, in the form of a gel or fluid, and to be delivered via the pore means to outside the body. In this way, the gel or fluid can be delivered to the anatomical surface of the patient that is in contact with the outer surface of the body in use.

In another embodiment, the further body, in the expanded configuration, is generally cylindrical in shape, comprising an internal passageway that defines the pharyngeal airway.

In another embodiment, the further body comprises an inner membrane, an outer membrane and a membrane volume in between, the outer membrane comprising pore means, the further body being such that when in the expanded configuration, any gel or fluid in the membrane volume is dispensed through the pore means to outside the further body. This allows an anesthetic, analgesic, medication or lubricant to be stored within the membrane volume, in the form of a gel or fluid, and to be delivered via the pore means to outside the further body. In this way, the gel or fluid can be delivered to the anatomical surface of the patient that is in contact with the outer surface of the further body in use.

Nasal Laryngeal Mask Airway—Device

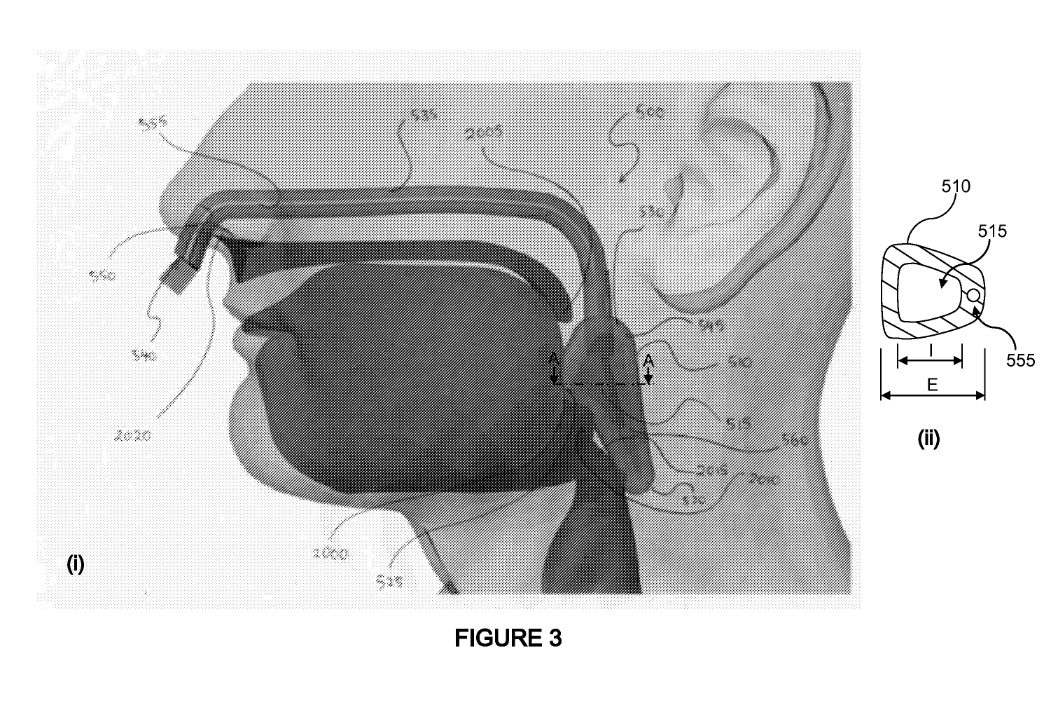

Referring to FIG. 3, a device 500 for maintaining an oropharyngeal airway 515 is shown. The device 500 comprises a body 510 adapted for configuration between a collapsed configuration (not shown) and an expanded configuration (shown in FIG. 3). The body 510, in the expanded configuration, is adapted for location in the oropharynx, to define the oropharyngeal airway 515, such that when the device 500 is fitted in a patient, and the body 510 is in the expanded configuration, the patient is able to breathe through the oropharyngeal airway 515 through his or her own respiratory effort. The body 510 in the expanded configuration is adapted to maintain the oropharyngeal airway 515 at least sufficiently open for breathing despite being subjected to an external pressure. The oropharyngeal airway 515 is substantially larger when the body 510 is in the expanded configuration than when the body 510 is in the collapsed configuration. Indeed, in this embodiment, the oropharyngeal airway 515 is undefined when the body 510 is in the collapsed configuration (not shown).

The device 500 for maintaining the oropharyngeal airway 515 can be used in a surgical context, for securing the patency of the patient's airway perioperatively, while the patient is anaesthetised or sedated, or postoperatively. PAP may be applied to the nasal side to ventilate the patient.

Pressure

The body 510, in the expanded position is adapted to maintain the oropharyngeal airway 515 at least sufficiently open for breathing when the body 510 is subjected to external pressure applied by the base of the tongue 2000, oropharyngeal wall, the posterior pharyngeal wall, the soft palate 2005, or any combination of the above. The body 510 sits in a supraglottic position below the soft palate 2005.

Hypopnea and apnea during anesthesia or sedation, is often caused by collapse of one or a combination of the above-mentioned tissues into the oropharynx causing obstruction of the natural airway. The device 500 for maintaining the oropharyngeal airway 515 is adapted to resist the collapse of the above-mentioned tissues in use, and maintain the oropharyngeal airway 515 at least sufficiently open for the patient to continue breathing.

Location

The body 510, in the expanded configuration, is adapted for location around at least a portion of the epiglottis 2010, glottis 2015 and laryngeal opening. In this embodiment, the body 510 is adapted for location around the epiglottis 2010, glottis 2015 and laryngeal opening, such that when the body 510 is located in the desired position in use and expanded into the expanded configuration, it is substantially fixed in place by its location around the epiglottis 2010, glottis 2015 and laryngeal opening.

Size & Shape

The length of the body 510 should be between 10 and 40 mm. In the preferred embodiment, the length of the body 510 is between 25 and 30 mm. The body 510, in the expanded configuration, is generally boot-shaped, such that, when the body 510 is correctly positioned in the patient's natural airway in use, the toe 520 of the boot-shape extends towards the patient's esophagus, the heel 525 of the boot-shape extends into the region between the base of the tongue 2000 and the epiglottis 2010, and the top 530 of the boot-shape is located within the oropharynx of the patient. In this embodiment, the toe 520 of the boot-shape substantially closes up the superior end of the esophagus such that emesis substances are substantially prevented from rising into the pharyngeal region of the patient's airway, thus reducing the likelihood of the aspiration of emesis substances into the patient's trachea in use.

In the expanded configuration and at the heel 525 of the boot-shape, the body 510 should have a maximum width of between 10 and 30 mm. In the preferred embodiment, in the expanded configuration and at the heel 525 of the boot, the body 510 has a maximum width of between 15 and 25 mm.

In another embodiment (not shown), in the expanded configuration, the body is generally kidney-shaped.

The body 510 in the expanded configuration is sized and shaped to allow the body 510 to comfortably and snugly fit into the region of a patient's airway at which the glottis 2015, epiglottis 2010, and laryngeal opening is located, and to provide sufficient pressure against collapse of the tissues of the patient's airway to maintain the airway at least sufficiently open for breathing. In practice, the body 510 is to be provided in various sizes to accommodate different patients. In one embodiment, the body 510 is sized to be received within a pediatric patient. In another embodiment, the body 510 is sized to be received within an adult patient.

The oropharyngeal airway 515 should have a minimum cross-sectional area of between 19 and 60 mm² when the body 510 is in the expanded configuration. In the preferred embodiment, the oropharyngeal airway 515 has a minimum cross-sectional area of between 20 and 50 mm² when the body 510 is in the expanded configuration. It is ideal that the oropharyngeal airway 515 is sufficiently large in use to facilitate breathing with minimal resistance. The body 510 is a generally thin-walled structure, which allows the cross-sectional area of the oropharyngeal airway 515 to be maximized. Nevertheless, the body 510, in the expanded configuration, is adapted to sufficiently withstand the application of external pressure from the collapsible tissues of the patient's natural airway in use.

Referring to FIGS. 3(i) and 3(ii), the generally boot-shaped body 510 in the expanded configuration defines an internal passageway that forms the oropharyngeal airway 515. In the expanded configuration, and generally at the heel 525 of the boot-shape, the body 510 should have an external diameter (E) of between 15 and 30 mm. In the preferred embodiment, in the expanded configuration, and generally at the heel 525 of the boot-shape, the body 510 has an external diameter (E) of between 15 and 25 mm

In the expanded configuration, and generally at the heel 525 of the boot-shape, the body 510 has an internal diameter (I) of generally between 5 and 12 mm, which defines the internal passageway for maintaining the oropharyngeal airway 515 at least sufficiently open for breathing. In this arrangement, the internal passageway through the body 510 has a corresponding internal cross-sectional area of between 20 and 120 mm², thereby satisfying the minimum cross-sectional area requirements described above for the oropharyngeal airway 515 when the body 510 is in the expanded configuration.

In a preferred embodiment, in the expanded configuration, and generally at the heel 530 of the boot-shape, the body 510 has an internal diameter of between 5 and 9 mm. In this arrangement, the internal passageway defined by the body 510 has a corresponding internal cross-sectional area of generally between 20 and 65 mm²,

In one embodiment (not shown), the device 500 for maintaining the oropharyngeal airway 515 comprises one or more fold lines to control movement of the body between the expanded and the collapsed configuration. In another embodiment (not shown), when the body is in the collapsed configuration it is twisted.

In another embodiment, when the body is in the expanded configuration, the oropharyngeal airway comprises an opening towards the nasopharynx and another opening towards the mouth and tongue. Thus, the body in the expanded configuration is adapted to maintain the oropharyngeal airway open both in a region of the oropharynx proximal to the nasopharynx of the patient, and in a region of the oropharynx of the patient proximal to the mouth of the patient.

Naso-Pharyngeal Airway Means

Referring to FIG. 3, the device 500 for maintaining the oropharyngeal airway 515 further comprises nasopharyngeal airway means 535 connected to the body 510 and defining a nasopharyngeal airway 540. The nasopharyngeal airway 540 is in fluid communication with the oropharyngeal airway 515. In the present embodiment, the nasopharyngeal airway means 535 is adapted for configuration between a collapsed and an expanded configuration. The nasopharyngeal airway means 535 is shown in FIG. 3 in the expanded configuration. In the collapsed configuration, the nasopharyngeal airway means 535 can be readily passed through one of the nares 2020, nasal cavity and nasopharynx and into the pharynx. In the expanded configuration, the patient is able to breathe through the nasopharyngeal airway 540, and through the oropharyngeal airway 515, by his or her own respiratory effort.

In another embodiment (not shown), the nasopharyngeal airway means is a flexible tube that is not configurable between a collapsed and an expanded configuration.

In one embodiment, the nasopharyngeal airway means 535 comprises a thermo-sensitive material such that at least one material property of the thermo-sensitive material, such as shape, changes as a function of temperature. In one embodiment, following insertion, the nasopharyngeal airway means 535 is adapted to absorb heat from the patient's tissues. Once sufficiently warm, the nasopharyngeal airway means 535 is adapted to change into a shape that better conforms to the natural contours of the nares 2020, nasal cavity and nasopharynx of the patient, making it more comfortable for the patient in use.

The nasopharyngeal airway means 535 should have an external diameter of between 6 and 12 mm. In the preferred embodiment, the nasopharyngeal airway means has an external diameter of between 7 and 10 mm. The nasopharyngeal airway means 535 is thus sufficiently narrow such that it is relatively comfortable for the patient when located in situ.

The nasopharyngeal airway 540 should have a minimum internal diameter of between 5 and 10 mm. In the preferred embodiment, the nasopharyngeal airway 540 has a minimum internal diameter of between 6 and 7 mm. The nasopharyngeal airway 540 should have a minimum cross-sectional area of between 15 and 80 mm². In the preferred embodiment, the nasopharyngeal airway 540 has a minimum cross-sectional area of between 25 and 50 mm². The internal diameter or internal cross-sectional area of the nasopharyngeal airway 540 is sufficiently large in use to facilitate breathing with minimal resistance.

In the case of the nasopharyngeal airway means 535 being manufactured from a solid material, the nasopharyngeal airway means 535 should have a wall thickness of between 0.3 and 10 mm. However, in the preferred embodiment, in which the nasopharyngeal airway means 535 is manufactured from a flexible material so as to be inflatable, the nasopharyngeal airway means 535 has a wall thickness of between 0.1 and 0.5 mm. The wall of the nasopharyngeal airway means 535 is optimized to maximize the internal cross-sectional area of the nasopharyngeal airway 540, while maintaining the nasopharyngeal airway clear, unobstructed and uncollapsed within the patient's nasal passage, nasopharynx and pharynx in use.

Inflation/Deflation

Referring to FIG. 3, the body 510 comprises an internal volume 545 that is fluid inflatable such that when inflated with fluid, the body 510 is in the expanded configuration and when deflated, the body 510 is in the collapsed configuration.

In the preferred embodiment, the nasopharyngeal airway means 535 is adapted to extend from the nares 2020 through the nasal cavity and nasopharynx and into the pharynx, providing a clear and unobstructed airway through which the patient is able to ventilate. The nasopharyngeal airway means 535 further comprises an inflation fluid passage 550 having a fluid port (not shown) adapted for connection to a fluid supply. The inflation fluid passage 550 is in fluid communication with the internal volume 545, such that the internal volume 545 can be readily inflated or deflated via the inflation fluid passage 540.

When the inflation fluid passage 540 is inflated, at least a portion of the length of the nasopharyngeal airway means is in the expanded configuration (as shown in FIG. 3) and when deflated, it is in the collapsed configuration.

In this way, the body 510 and the nasopharyngeal airway means 535 are both adapted to be inflated through the inflation fluid passage 550 and by the same fluid supply. In the preferred embodiment, the body 510 and nasopharyngeal airway means 535 move to the expanded configuration when a fluid pressure is applied at the fluid port and to the collapsed configuration when the fluid is withdrawn through the fluid port. Therefore constant fluid pressure is not required to maintain the body 515 and nasopharyngeal airway means 535 in the expanded configuration.

In one embodiment, the fluid port comprises an airway connector, and is thus adapted for connection to an air supply. In the preferred embodiment, the airway connector is a standard 22 mm airway connector.

In one embodiment, the inflation fluid passage is a lumen. In the preferred embodiment, the nasopharyngeal airway means 535 is a dual, thin-walled structure, the inflation fluid passage 550 provided by the volume between outer and inner walls of the dual, thin-walled structure. Thus, when viewed in cross-section, the inflation fluid passage 550 circumferentially surrounds the nasopharyngeal airway 540. Having the inflation fluid passage located between the inner and outer walls minimizes interruption within the nasopharyngeal airway in comparison with locating the inflation fluid passage within the nasopharyngeal airway itself, thereby minimizing the resistance to breathing of the user in use.

The inflation fluid passage 550 should have an internal cross-sectional area of between 0.5 and 2 mm². In the preferred embodiment, the inflation fluid passage 550 has an internal cross-sectional area of between 1 and 1.5 mm². The inflation fluid passage 550 has an internal cross-sectional area that is sufficiently large such that a fluid may be conducted within the inflation fluid passage 550 with minimal resistance. Nevertheless, the inflation fluid passage 550 has a sufficiently small internal cross-sectional area to allow the cross-sectional area of the nasopharyngeal airway 540 to be maximized within the nasopharyngeal airway means 535.

In another embodiment, the inflation fluid passage is one or more inflation fluid sub-passages. It may be advantageous for each sub-passage to be separately inflatable, such that the compromise of one sub-passage due to puncture or otherwise will not jeopardise the functionality of the entire device.

In one embodiment, the fluid supply is a pressurised fluid supply, enabling spontaneous inflation of the nasopharyngeal airway means 535 and the internal volume 545 once the fluid port of the inflation fluid passage 550 is engaged with the fluid supply.

In one embodiment, the nasopharyngeal airway means 535 comprises an outer surface that is adapted to slowly release an anesthetic substance. Release of a local anesthetic to the tissues of the nasal cavity, nasopharynx and pharynx with which the nasopharyngeal airway means 535 comes into contact minimizes discomfort and soreness to the patient. In one embodiment, the outer surface of the nasopharyngeal airway means 535 is impregnated with the anesthetic substance, facilitating slow release of the anesthetic substance to the tissues with which the outer surface of the nasopharyngeal airway means 535 comes into contact.

In another embodiment, the nasopharyngeal airway means 535 comprises a plurality of micropores and/or nanopores extending from the inflation fluid passage 550 to outside the nasopharyngeal airway means 535. The micropores and/or nanopores can be adapted to duct and slowly release of the anesthetic substance to the tissues with which the nasopharyngeal airway means 535 comes into contact.

In one embodiment, the body 510 comprises an outer surface that is adapted to slowly release an anesthetic substance. Release of a local anesthetic to the tissues of the airway with which the body 510 comes into contact minimizes discomfort and soreness to the patient, and prevents the potential stimulation of a potential gag reflex in use.

In one embodiment, the outer surface of the body 510 is impregnated with the anesthetic substance prior to insertion, facilitating slow release of the anesthetic substance to the tissues of the patient's airway with which the outer surface of the body 510 comes into contact. In another embodiment, the body 510 comprises a plurality of micropores and/or nanopores extending from the internal volume 545 to outside the body 510. The micropores and/or nanopores can be adapted to duct and slowly release of the anesthetic substance to the tissues of the patient's airway with which the body 510 comes into contact.

In one embodiment, the inflation fluid passage 550 is adapted to conduct a gas, such as air.

Air supplies are readily available and cost-effective. In another embodiment, the inflation fluid passage 550 is adapted to conduct a liquid, such as water. Water is also readily available and cost-effective. In another embodiment, an anesthetic gas, or an anesthetic liquid is conducted along the inflation fluid passage 550 to inflate the internal volume 545. The presence of micropores and/or nanopores in the surface of the body 510, and/or in the surface of the nasopharyngeal airway means 535, would enable the anesthetic substance to be ducted to the surface of the body 510 and/or the nasopharyngeal airway means 535 to provide anesthetic relief to the tissues of the patient with which the body 510 and/or nasopharyngeal airway means 535 come into contact.

Guide Means

In the present embodiment, the device 500 for maintaining the oropharyngeal airway 515 further comprises guide means 555 connected to the body. The guide means 555 is a nasopharyngeal guide means adapted to direct the body 510 in the collapsed configuration through one of the nares 2020, nasal cavity and nasopharynx and into the pharynx. Thus, the body 510 is readily inserted and directed into the desired position in the patient's pharynx. If correctly inserted, the patient will feel minimal discomfort during and following the insertion procedure, and will not sustain any damage to the nares 2020, nasal cavity, nasopharynx or pharynx. Furthermore, insertion of the device 500 and the location of the device 500 in situ is relatively comfortable and well-tolerated, allowing the device 500 to be inserted while the patient is awake. This allows the patient's airway to be secured prior to the induction of anesthesia or sedation, reducing the likelihood of difficult intubation, and of anesthesia-related mortality and morbidity caused by loss of the patency of the airway. The device 500 for maintaining the oropharyngeal 515 is particularly useful for maxillofacial or oral procedures because it allows the patient's airway to be secured with perfect oral access.

The guide means 555 is adapted to direct the body 510 such that it does not contact, or at least does not apply a substantial pressure to the region of the turbinate at the entrance to the nasopharynx. The region of the turbinate is a sensitive region, and thus, the body 510 can be inserted and directed into place without the patient experiencing excessive discomfort or suffering damage in the region of the turbinate.

In the preferred embodiment, the guide means 555 is flexible and pliable, but sufficiently semi-rigid such that it facilitates ease of insertion of the body 510 into the pharynx. In one embodiment, the length of the guide means 555 is bendable into different shapes.

In another embodiment, the guide means 555 comprises a thermo-sensitive material such that at least one material property of the thermo-sensitive material, such as shape, changes as a function of temperature. In one embodiment, following insertion, the guide means 555 is adapted to absorb heat from the patient's tissues. Once sufficiently warm, the guide means 555 is adapted to change into a shape that better conforms to the natural contours of the nares 2020, nasal cavity and nasopharynx of the patient, making it more comfortable for the patient in use.

In another embodiment, the guide means 555 comprises a shape memory material and is bendable into a new shape when heated and retains the new shape when cooled. An advantage of this embodiment is that it allows the guide means 555 to be tailored to shape to better conform to the natural contours of the nares 2020, nasal cavity and nasopharynx of each individual patient, making it more comfortable for the patient in use.

In another embodiment, the guide means 555 comprises a cord. The cord may be adapted to be long enough to be located external to the patient's nares 2020 in use such that retraction of the device 500 is readily achieved when desired.

The guide means 555 should have an external cross-sectional area of between 1.5 and 4 mm². In the preferred embodiment, the guide means 555 has an external cross-sectional area of between 2 and 3.5 mm². The guide means 555 is thus sufficiently narrow such that it is comfortable for the patient and facilitates minimal interference with the breathing of the patient in use, yet facilitates easy guidance of the body 510 into the desired position in the patient's pharynx.

In one embodiment, the guide means comprises one or more bend regions, each bend region being adapted to move between a first angle and a second angle. This allows mechanical manipulation of the guide means to be limited to a pre-defined range of movement, which prevents the guide means from being bent out of shape or damaged.

In another embodiment, at least one of the bend regions is adapted to move between the first angle and the second angle upon application of an electric current. In another embodiment, the at least one bend region comprises an electro-active shape memory polymer element and is adapted to move between the first angle and the second angle upon application of an electric current to the electro-active shape memory polymer element. In one embodiment, the electro-active shape memory polymer element is a piezo-electric polymer element.

In another embodiment, at least one of the bend regions is adapted to move between the first angle and the second angle upon application of light within a predetermined wavelength range. In another embodiment, the at least one bend region comprises a light-induced shape memory polymer element and is adapted to move between the first angle and the second angle upon application of a light within a predetermined wavelength range to the light-induced shape memory polymer element.

In another embodiment, the at least one bend region comprises an electromechanical servo motor and is adapted to move between the first angle and the second angle upon application of an electric current to the electromechanical servo motor.

In another embodiment, the guide means comprises a series of elements connected by hinge means, and a tensioning means, the guide means being adapted to move between a first configuration in which the series of elements are disposed relative to each other in a first disposition and a second configuration in which the series of elements are disposed relative to each other in a second disposition by tensioning of the tensioning means. In one embodiment, the series of elements are hollow and the tensioning means is a tensioning cord that extends through the series of elements.

In another embodiment, the guide means comprises a series of elements connected by hinge means, and a push means, the guide means being adapted to move between a first configuration in which the series of elements are disposed relative to each other in a first disposition and a second configuration in which the series of elements are disposed relative to each other in a second disposition by pushing of the push means. In another embodiment, the series of elements are hollow and the push means extends through the series of elements.

In another embodiment, the guide means comprises a sheath means and a rotational member located therein, the rotational member being connected to the body at one end and extending externally from the sheath means at the other end, such that when the rotational member is rotated relative to the sheath means in a first direction the body twists to the collapsed configuration and when the rotational member is rotated relative to the sheath means in the other direction the body un-twists to the expanded configuration.

In another embodiment, the guide means comprises a sheath means and a push-pull member located therein, the push-pull member being connected to the body at one end and extending externally from the sheath means at the other end, such that when the push-pull member is moved relative to the sheath means in one direction the body collapses to the collapsed configuration and in the other direction the body expands to the expanded configuration.

Advantageously, the guide means 555 can be adapted to be mechanically manipulated by any one or more of a range of different means, to dispose it in a first configuration to optimize it for ease of insertion, and in a second configuration to optimize it for comfort to the patient in situ or to change the shape of the guide means 555 during insertion to reduce impingement of the device on anatomical surfaces.

Referring to FIG. 3, a proximal end 560 of the guide means 555 comprises a generally cone-shaped tip. In this embodiment, the proximal end 560 of the guide means 555 is more specifically described as a blunted and frusto-conical tip. The texture of the outer surface of the proximal end 560 of the guide means 555 is smooth. Thus, the proximal end 560 is adapted to cause no damage and minimal discomfort to the patient during insertion and when located in situ. Furthermore, the proximal end 560 of the guide means 555 is weighted so that it tends to drop down, helping to maintain the body 510 in the desired position and orientation in use.

In one embodiment (not shown), a distal end of the guide means comprises an anchor. Thus, the distal end of the guide means is adapted to remain external to the patient's nasal cavity, and to be affixed to an external region of the patient's facial anatomy to help secure the body in the desired position in use. In another embodiment, the guide means is adapted to be of a pre-determined length and to terminate in the anchor. The anchor, once attached to a part of the patient's facial anatomy, limits any further insertion of the guide means than is desired, and as previously described, helps maintain the body in the desired position in use. Thus, correct positioning of the body into the patient's pharynx is easily ascertainable. The length of the guide means can be tailored to suit each individual patient.

In one embodiment, the anchor is one or more wings that contact an outer surface of at least one of the nares 2020 in use. In another embodiment, the anchor is a clip adapted for clipping onto at least one of the nares 2020 in use. In another embodiment, the anchor comprises a face mask, nasal mask, head band or head gear. In another embodiment, the anchor is adapted to engage the septum. In any one of the embodiments of the anchor, the anchor is adapted to comfortably fix the guide means to minimize movement of the guide means within the nares 2020, nasal cavity and nasopharynx, thereby minimising discomfort to the patient and/or damage to the patient's tissues in use.

A cross-section of a main length of the guide means 555 should fit within a diameter of at most 2.5 mm. In the preferred embodiment the cross-section of a main length of the guide means 555 fits within a diameter of 2 mm. The guide means 555 is sufficiently narrow such that it is comfortable for the patient and facilitates minimal interference with the breathing of the patient in use, yet facilitates easy guidance of the body 510 into the desired position in the pharynx.

In one embodiment, the nasopharyngeal airway means comprises a nasopharyngeal guide means adapted to direct the body in the collapsed configuration through the nares, nasal cavity and nasopharynx and into the pharynx. Thus, in this way, the body is easily and readily inserted and directed into the desired position. If correctly inserted, the patient will feel minimal discomfort, and will not sustain any damage to the nares 2020, nasal cavity, nasopharynx or pharynx.

Materials

In the preferred embodiment, the body 510, the nasopharyngeal airway means 535 and the guide means 555 are made of a biocompatible material, such that the device 500, in use, elicits little or no immune response from the patient.

In one embodiment, the guide means 555 is made of plastic. More specifically, the guide means 555 is made from a plastic out of the group of plastics comprising polyvinyl chloride, polyethylene, PEEK, Ultem PEI, Polysulfone, polypropylene and polyurethane. As described above, the guide means 555 is adapted to be flexible, pliable, semi-rigid, and/or bendable into different shapes. In another embodiment, the guide means is made of silicone.

In one embodiment, the body 510 is made of plastic. More specifically, the body 510 is made from a plastic out of the group of plastics comprising polyvinyl chloride, polyethylene, PEEK, Ultem PEI, Polysulfone, polypropylene and polyurethane. In another embodiment, the body 15 is made of silicone, which allows the body 510 to be sufficiently elastic such that it is inflatable and is comfortable for the patient in use.

In one embodiment, the nasopharyngeal airway means 535 is made of plastic. More specifically, the nasopharyngeal airway means 535 us made from a plastic out of the group of plastics comprising: polyvinyl chloride, polyethylene, PEEK, Ultem PEI, Polysulfone, polypropylene and polyurethane. In another embodiment, the nasopharyngeal airway means 535 is made of silicone, which allows the nasopharyngeal airway means 535 to be sufficiently elastic such that it is inflatable and is comfortable for the patient in use. In the preferred embodiment, the nasopharyngeal airway means 535 is a dual, thin-walled structure, the inflation fluid passage provided by the volume between outer and inner walls of the dual, thin-walled structure.

In one embodiment, the body 510 comprises a foam portion that is adapted to be compressed when the body is in the collapsed configuration. When the body is expanded to the expanded configuration, the foam portion becomes uncompressed and helps resist the collapse of the patient's airway in use.

Other Features

In one embodiment, in the expanded configuration, an outer surface of the body comprises one or more grooves adapted to allow fluids to drain when the outer surface is in contact with an anatomical surface in use. In another embodiment, in the expanded configuration, an outer surface of the nasopharyngeal airway means comprises one or more grooves adapted to allow fluids to drain when the outer surface is in contact with an anatomical surface in use.

The grooves prevent the buildup of fluids produced by the patient in the nasopharynx or pharynx in use.

In one embodiment, an outer surface of at least a portion of the guide means is adapted to slowly release an anesthetic substance in use, to minimize discomfort and soreness and prevent the stimulation of a potential gag reflex in use. In one embodiment, the outer surface of the guide means is adapted to be impregnated with the anesthetic substance, which is an effective way to facilitate slow release of the anesthetic substance.

In one embodiment, the body is adapted to move between the expanded and collapsed configuration on application of an electric current. In another embodiment, the body comprises one or more electro-active polymer elements and is adapted to move between the expanded and collapsed configuration on application of an electric current to the one or more electro-active polymer elements. In one embodiment, the nasopharyngeal airway means comprises a wire adapted for connection to a power source at one end and to the one or more electro-active polymer elements at the other end, allowing the application of an electric current to be safely and effectively performed.

Thus, the body and/or nasopharyngeal airway means are configurable between the expanded and collapsed configurations by one or more of a range of means.

In another embodiment, the body comprises an inner membrane, an outer membrane and a membrane volume in between, the outer membrane comprising pore means, the body being such that when in the expanded configuration, any gel or fluid in the membrane volume is dispensed through the pore means to outside the body. This allows an anesthetic, analgesic, medication or lubricant may be stored within the membrane volume, in the form of a gel or fluid, and to be delivered via the pore means to outside the body. In this way, the gel or fluid can be delivered to the anatomical surface of the patient that is in contact with the outer surface of the body in use.

In another embodiment, the device for maintaining an oropharyngeal airway further comprises oesophageal fluid canal means defining an oesophageal fluid canal. In this embodiment, a proximal end of the oesophageal fluid canal comprises an opening towards the esophagus and a distal end is located outside of the nares 2020 of the patient in use.

Thus, the oesphageal fluid canal is adapted to conduct emesis substances from the esophagus, from the proximal end to the distal end of the canal, thereby preventing aspiration of the emesis substances into the patient's airway in use.

Oropharyngeal Cushion

According to a third embodiment of the present invention, a method of creating an oropharyngeal airway is further described. The method of creating an oropharyngeal airway comprises the steps of:
inserting a guide means through one of the nares, nasal cavity and nasopharynx and into the pharynx to locate an expandable body in a collapsed configuration in the pharynx, adjacent the base of the tongue above the cartilaginous epiglottis; and
expanding the expandable body from the collapsed configuration to an expanded configuration to create the oropharyngeal airway through the expandable body.

The expandable body is adapted to prevent the patient's pharynx from collapsing in the region of the oropharynx that is adjacent the base of the tongue, and to create an oropharyngeal airway through which the patient is able to ventilate.

The method of creating an oropharyngeal airway can be used in the treatment of obstructive sleep apnea. The method can also be used in a surgical context for securing the patency of the patient's airway perioperatively, while the patient is anaesthetized or sedated, or postoperatively.

The expandable body is adapted to be easily manoeuvred into the patient's airway in the collapsed configuration, and once located in the desired position within the patient's airway, to be deployed into the expanded configuration. The method of allows the expandable body to be easily and readily inserted and directed into the desired position by aid of the guide means, such that the patient feels minimal discomfort, and does not sustain damage to the nares, nasal cavity, nasopharynx or pharynx. This method is readily performed on an awake patient, which allows the patient's airway to be secured prior to the induction of sedation or anesthesia, thus reducing the likelihood of difficult intubation, and of anesthesia-related mortality and morbidity caused by loss of the patency of the airway. The method of creating an oropharyngeal airway can be easily learnt and readily employed by a person who is not a medical practitioner, including the patient himself or herself.

Furthermore, the method of creating an oropharyngeal airway allows the intake of air into the patient's airways to be naturally humidified and filtered by the patient's own nasal passages and/or oral passage in use.

In the preferred embodiment, the guide means comprises an inflation passage, the body being in fluid communication with the inflation passage and inflatable from the collapsed configuration to the expanded configuration, and the step of expanding the expandable body comprises the step of:
delivering a fluid through the inflation passage to the body to inflate the body from the collapsed configuration to the expanded configuration.

This method of creating an oropharyngeal airway thus allows for the body to be easily configurable between the expanded and collapsed configurations by inflating and deflating the body respectively.

In one embodiment, the fluid is a gas, such as air or an anesthetic gas. Air supplies are readily available and cost-effective. Anesthetic gas can be used to provide local anesthetic to minimize discomfort to the patient.

In another embodiment, the fluid is a liquid, such as water or an anesthetic liquid. Water supplies are also readily available and cost-effective. Anesthetic liquid can be used to provide local anesthetic to minimize discomfort to the patient.

### Nasal Laryngeal Mask Airway—Method

According to a fourth embodiment of the present invention, a method of creating an oropharyngeal airway is described. The method of creating an oropharyngeal airway comprises the steps of:
inserting a nasopharyngeal airway means in a collapsed configuration through one of the nares, nasal cavity and nasopharynx and into the pharynx to locate an expandable body in a collapsed configuration in the pharynx substantially around the glottis, epiglottis and laryngeal opening; and
expanding the nasopharyngeal airway means and the expandable body from the collapsed configuration to an expanded configuration to create the oropharyngeal airway through the nasopharyngeal airway means and expandable body.

The expandable body is adapted to prevent the patient's pharynx from collapsing, and to create an oropharyngeal airway and nasopharyngeal airway through which the patient is able to ventilate.

The method of creating an oropharyngeal airway can be used in a surgical context, for securing the patency of the patient's airway perioperatively, while the patient is anaesthetised or sedated, or postoperatively.

The expandable body is easily manoeuvred into the patient's airway in the collapsed configuration such that the patient feels minimal discomfort, and does not sustain damage to the nares, nasal cavity, nasopharynx or pharynx. Once located in the desired position within the patient's airway, the expandable body is adapted to be deployed into the expanded configuration. This method is readily performed on an awake patient, and thus the patient's airway is advantageously secured prior to the induction of sedation or anesthesia, reducing the likelihood of difficult intubation, and of anesthesia-related mortality and morbidity caused by loss of the patency of the airway.

In the preferred embodiment, the nasopharyngeal airway means comprises an inflation passage that is inflatable from the collapsed configuration to the expanded configuration, and the body, being inflatable from the collapsed configuration to the expanded configuration, is in fluid communication with the inflation passage. The step of expanding the nasopharyngeal airway means and the expandable body comprises the step of:
delivering a fluid into the inflation passage and thus also into the body to inflate the nasopharyngeal airway means and the body from the collapsed configuration to the expanded configuration.

The method of creating an oropharyngeal airway allows for the nasopharyngeal airway means and the expandable body to be easily configurable between the expanded and collapsed configurations by inflating and deflating the nasopharyngeal airway means and the expandable body, respectively.

In one embodiment, the fluid is a gas, such as air or an anesthetic gas. Air supplies are readily available and cost-effective. Anesthetic gas can be used to provide local anesthetic to minimize discomfort to the patient.

In another embodiment, the fluid is a liquid, such as water or an anesthetic liquid. Water supplies are also readily available and cost-effective. Anesthetic liquid can be used to provide local anesthetic to minimize discomfort to the patient.

System

According to a fifth embodiment of the present invention, a system for maintaining an oropharyngeal airway is described, comprising:
a device for maintaining an oropharyngeal airway;
further comprising a fluid port, the fluid port being in fluid communication with the internal volume; and
an inflation means connected to the fluid port and adapted to deliver a fluid into the internal volume of the body.

In one embodiment, the inflation means is a piston-cylinder arrangement. In one embodiment, the piston-cylinder arrangement is a syringe. Syringes are portable, easy to use, economically produced, and do not require connection to an electrical power source.

In another embodiment, the inflation means is an electrically powered air pump, which facilitates fast and efficient inflation of the body to the expanded configuration.

In another embodiment, the inflation means is a finger operated air pump, and is thus portable, easy to use, economically produced, and does not require connection to an electrical power source.

In another embodiment, the inflation means is further adapted to withdraw the fluid from the internal volume of the body. This feature conveniently allows inflation and deflation to be achieved via the same means.

In one embodiment, the fluid is a gas, such as air or an anesthetic gas. Air supplies are readily available and cost-effective. Anesthetic gas can be used to provide local anesthetic to minimize discomfort to the patient.

In another embodiment, the fluid is a liquid, such as water or an anesthetic liquid. Water supplies are also readily available and cost-effective. Anesthetic liquid can be used to provide local anesthetic to minimize discomfort to the patient.

In one embodiment, the system for maintaining an oropharyngeal airway further comprises an overpressurisation valve adapted to open and release pressure from the internal volume when the pressure is higher than a predefined pressure limit. Thus, the system is adapted to detect when the internal volume is overpressurised and to spontaneously remedy the overpressurisation if and when it is detected, thereby allowing the device to be used with a high level of safety in the patient.

Other embodiments of the invention are also disclosed.

Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the medical and medical devices industry.

The claims defining the invention are as follows:

1. A device for maintaining an oropharyngeal airway, comprising:
a body adapted for configuration between a collapsed and an expanded configuration,
and guide means being a nasopharyngeal guide means adapted to direct the body in the collapsed configuration through one of the nares, nasal cavity and nasopharynx and into the pharynx;
wherein in the expanded configuration, the body is generally torus shaped and adapted for location in the oropharynx, to define an oropharyngeal airway and to maintain the oropharyngeal airway at least sufficiently open for breathing when the body is subjected to an external pressure, the oropharyngeal airway being substantially larger when the body is in the expanded configuration than when the body is in the collapsed configuration

2. A device as claimed in claim 1, wherein the external pressure is applied by the base of the tongue.

3. A device as claimed in claim 1, wherein the external pressure is applied by the oropharyngeal wall.

4. A device as claimed in claim 1, wherein in the expanded configuration, the body is adapted to apply a pressure to one or more areas from the following group of areas:
(i) the oropharyngeal wall;
(ii) the posterior pharyngeal wall;
(iii) the base of the tongue; and
(iv) the soft palate.

5. A device as claimed in claim 1, wherein the body is adapted for location at a location upstream of the cartilaginous epiglottis.

6. A device as claimed in claim 5, wherein at the location, the body is not in contact with the cartilaginous epiglottis.

7. A device as claimed in claim 1, wherein the body has a length of between 10 and 40 mm.

8. A device as claimed in claim 7, wherein the body has a length of between 20 and 35 mm.

9. A device as claimed in claim 8, wherein the body has a length of between 25 and 30 mm.

10. A device as claimed in claim 1, wherein in the expanded configuration, the body has a maximum width of between 5 and 12 mm.

11. A device as claimed in claim 10, wherein in the expanded configuration, the body has a maximum width of between 6 and 10 mm.

12. A device as claimed in claim 1, wherein in the collapsed configuration, the body fits within a diameter of 10 mm.

13. A device as claimed in claim 12, wherein in the collapsed configuration, the body fits within a diameter of 7 mm.

14. A device as claimed in claim 13, wherein in the collapsed configuration, the body fits within a diameter of about 6 mm.

15. A device as claimed in claim 1, wherein the oropharyngeal airway has a minimum cross-sectional area of between 6 and 13 mm² when the body is in the expanded configuration.

16. A device as claimed in claim 15, wherein the oropharyngeal airway has a minimum cross-sectional area of between 7 and 9 mm² when the body is in the expanded configuration.

17. A device as claimed in claim 17, wherein in the expanded configuration, the body has an external diameter of between 5 mm and 12 mm.

18. A device as claimed in claim 18, wherein the external diameter is between 6 mm and 10 mm.

19. A device as claimed in claim 17, wherein in the expanded configuration, the body has an internal diameter of between 4 mm and 9 mm.

20. A device as claimed in claim 20, wherein the internal diameter is between 4 mm and 8 mm.

21. A device as claimed in claim 1, wherein the body is a generally thin-walled structure.

22. A device as claimed in claim 22, comprising one or more fold lines to control movement of the body between the expanded and the collapsed configuration.

23. A device as claimed in claim 1, wherein when the body is in the collapsed configuration it is twisted.

24. A device as claimed in claim 1, wherein when the body is in the expanded configuration, the oropharyngeal airway comprises an opening towards the nasopharynx and another opening towards the mouth and tongue.

25. A device as claimed in claim 1, wherein the body is sized to be received within a pediatric patient.

26. A device as claimed in claim 1, wherein the body is sized to be received within an adult patient.

27. A device as claimed in claim 1, further comprising guide means connected to the body.

28. A device as claimed in claim 1, wherein the guide means is adapted to direct the body such that it does not apply a substantial pressure to the region of the turbinate at the entrance to the nasopharynx.

29. A device as claimed in claim 1, wherein the guide means is adapted to direct the body such that it does not contact the region of the turbinate at the entrance to the nasopharynx.

30. A device as claimed in claim 1, wherein the guide means is connected to a region of the body that is posterior to the oropharyngeal airway in use when the body is in the expanded configuration.

31. A device as claimed in claim 1, wherein the guide means is flexible.

32. A device as claimed in claim 1, wherein the guide means is semi-rigid.

33. A device as claimed in claim 1, wherein the guide means is pliable.

34. A device as claimed in claim 1, wherein the length of the guide means is bendable into different shapes.

35. A device as claimed in claim 1, wherein the guide means comprises a thermo-sensitive material such that at least one material property of the thermo-sensitive material changes as a function of temperature.

36. A device as claimed in claim 3, wherein the thermo-sensitive material changes shape as a function of temperature such that the guide means changes shape as a function of temperature.

37. A device as claimed in claim 1, wherein the guide means comprises a shape memory material and is bendable into a new shape when heated and retains the new shape when cooled.

38. A device as claimed in claim 1, wherein the guide means comprises a cord.

39. A device as claimed in claim 1, wherein the guide means has an external cross-sectional area of between 0.5 and 3 mm².

40. A device as claimed in claim 39, wherein the guide means has an external cross-sectional area of between 1 and 2 mm².

41. A device as claimed in claim 1, wherein the guide means comprises one or more bend regions, each bend region being adapted to move between a first angle and a second angle.

42. A device as claimed in claim 41, wherein at least one of the bend regions is adapted to move between the first angle and the second angle upon application of an electric current.

43. A device as claimed in claim 434, wherein the at least one bend region comprises an electro-active shape memory polymer element and is adapted to move between the first angle and the second angle upon application of an electric current to the electro-active shape memory polymer element.

44. A device as claimed in claim 44, wherein the electro-active shape memory polymer element is a piezo-electric polymer element.

45. A device as claimed in claim 41, wherein at least one of the bend regions is adapted to move between the first angle and the second angle upon application of light within a predetermined wavelength range.

46. A device as claimed in claim 45, wherein the at least one bend region comprises a light-induced shape memory polymer element and is adapted to move between the first angle and the second angle upon application of a light within a predetermined wavelength range to the light-induced shape memory polymer element.

47. A device as claimed in claim 424, wherein the at least one bend region comprises an electromechanical servo motor and is adapted to move between the first angle and the second angle upon application of an electric current to the electromechanical servo motor.

48. A device as claimed in claim 1, wherein the guide means comprises a series of elements connected by hinge means, and a tensioning means, the guide means being adapted to move between a first configuration in which the series of elements are disposed relative to each other in a first disposition and a second configuration in which the series of elements are disposed relative to each other in a second disposition by tensioning of the tensioning means.

49. A device as claimed in claim 48, wherein the series of elements are hollow and the tensioning means is a tensioning cord that extends through the series of elements.

50. A device as claimed in claim 1, wherein the guide means comprises a series of elements connected by hinge means, and a push means, the guide means being adapted to move between a first configuration in which the series of elements are disposed relative to each other in a first disposition and a second configuration in which the series of elements are disposed relative to each other in a second disposition by pushing of the push means.

51. A device as claimed in claim 5, wherein the series of elements are hollow and the push means extends through the series of elements.

52. A device as claimed in claim 1, wherein the guide means comprises a sheath means and a rotational member located therein, the rotational member being connected to the body at one end and extending externally from the sheath means at the other end, such that when the rotational member is rotated relative to the sheath means in a first direction the body twists to the collapsed configuration and when the rotational member is rotated relative to the sheath means in the other direction the body un-twists to the expanded configuration.

53. A device as claimed in claim 1, wherein the guide means comprises a sheath means and a push-pull member located therein, the push-pull member being connected to the body at one end and extending externally from the sheath means at the other end, such that when the push-pull member is moved relative to the sheath means in one direction the body collapses to the collapsed configuration and in the other direction the body expands to the expanded configuration.

54. A device as claimed in claim 1, wherein a proximal end of the guide means, being proximal to a patient in use, comprises a generally cone-shaped tip.

55. A device as claimed in claim 54, wherein the proximal end of the guide means is blunted.

56. A device as claimed in claim 55, wherein the proximal end of the guide means is generally frusto-conical.

57. A device as claimed in claim 54, wherein the proximal end of the guide means has a smooth outer surface.

58. A device as claimed in claim 1, wherein a proximal end of the guide means, being proximal to a patient in use, is weighted so it tends to drop down.

59. A device as claimed in claim 1, wherein a distal end of the guide means, being distal to a patient in use, comprises an anchor.

60. A device as claimed in claim 59, wherein the anchor is one or more wings that contact an outer surface of at least one of the nares in use.

61. A device as claimed in claim 59, wherein the anchor is a clip adapted for clipping onto at least one of the nares in use.

62. A device as claimed in claim 59, wherein the anchor comprises a face mask, nasal mask, head band or head gear.

63. A device as claimed in claim 59, wherein the anchor is adapted to engage the septum.

64. A device as claimed in claim 1, wherein a cross-section of a main length of the guide means fits within a diameter of 1 mm.

65. A device as claimed in claim 64, wherein a cross-section of a main length of the guide means fits within a diameter of 2 mm.

66. A device as claimed in claim 1, wherein the guide means defines a fluid passage.

67. A device as claimed in claim 66, wherein the fluid passage is an inflation passage and has an internal cross-sectional area of between 1 and 3 mm².

68. A device as claimed in claim 67, wherein the inflation passage has an internal cross-sectional area of between 1.5 and 2 mm².

69. A device as claimed in claim 66, wherein the guide means comprises a lumen, the lumen defining the fluid passage.

70. A device as claimed in claim 66, wherein the body comprises an internal volume that is fluid inflatable such that when inflated with fluid the body is in the expanded configuration and when deflated the body is in the collapsed configuration.

71. A device as claimed in claim 70, wherein the fluid passage is in fluid communication with the internal volume.

72. A device as claimed in claim 66, wherein a distal end of the fluid passage, being distal to a patient in use, comprises a port adapted for connection to a fluid supply.

73. A device as claimed in claim 72, wherein the fluid supply is a pressurized fluid supply.

74. A device as claimed in claim 70, wherein the body comprises an outer surface that is adapted to slowly release an anesthetic substance.

75. A device as claimed in claim 74, wherein the outer surface is adapted to be impregnated with the anesthetic substance.

76. A device as claimed in claim 74, wherein the body comprises a plurality of micropores extending from the internal volume to outside the body.

77. A device as claimed in claim 74, wherein the body comprises a plurality of nanopores extending from the internal volume to outside the body.

78. A device as claimed in claim 70, wherein the fluid is a gas.

79. A device as claimed in claim 78, wherein the fluid is air.

80. A device as claimed in claim 78, wherein the fluid comprises an anesthetic gas.

81. A device as claimed in claim 70, wherein the fluid is a liquid.

82. A device as claimed in claim 81, wherein the fluid is water.

83. A device as claimed in claim 81, wherein the fluid comprises an anesthetic liquid.

84. A device as claimed in claim 1, wherein the body and the guide means are made of a biocompatible material.

85. A device as claimed in claim 84, wherein the guide means is made of plastic.

86. A device as claimed in claim 85, wherein the guide means is made from a plastic out of the group of plastics comprising: polyvinyl chloride, polyethylene, PEEK, Ultem PEI, Polysulfone, polypropylene and polyurethane.

87. A device as claimed in claim 84, wherein the guide means is made of silicone.

88. A device as claimed in claim 84, wherein the body is made of plastic.

89. A device as claimed in claim 88, wherein the body is made from a plastic out of the group of plastics comprising: polyvinyl chloride, polyethylene, PEEK, Ultem PEI, Polysulfone, polypropylene and polyurethane.

90. A device as claimed in claim 84, wherein the body is made of silicone.

91. A device as claimed in claim 1, wherein the body comprises a foam portion that is adapted to be compressed when the body is in the collapsed configuration.

92. A device as claimed in claim 71, further comprising a further body adapted for configuration between a collapsed and an expanded configuration,
wherein in the expanded configuration, the further body is adapted for location in the nasopharynx or upper oropharynx, to define a pharyngeal airway and to maintain the pharyngeal airway at least sufficiently open for breathing when the further body is subjected to an additional external pressure,
the pharyngeal airway being substantially larger when the further body is in the expanded configuration than when the further body is in the collapsed configuration,
the further body being connected to the guide means,
the guide means being adapted to direct the further body through one of the nares and nasal cavity,
the further body comprising a further internal volume that is fluid inflatable such that when inflated with fluid the further body is in the expanded configuration and when deflated the further body is in the collapsed configuration.

93. A device as claimed in claim 92, wherein the fluid passage is in fluid communication with the further internal volume of the further body.

94. A device as claimed in claim 92, wherein the additional external pressure is applied by the posterior region of the soft palate.

95. A device as claimed in claim 92, wherein the additional external pressure is applied by the nasopharyngeal wall.

96. A device as claimed in claim 92, wherein the additional external pressure is applied by the oropharyngeal wall.

97. A device as claimed in claim 92, wherein in the expanded configuration, the further body is adapted to apply a pressure to one or more areas from the following group of areas:
(v) the nasopharyngeal wall;
(vi) the oropharyngeal wall;
(vii) the posterior pharyngeal wall; and
(viii) the posterior region of the soft palate.

98. A device as claimed in claim 92, wherein the further body is adapted for location at a location upstream of the body in use.

99. A device as claimed in claim 98, wherein at the location in use, the further body is not in contact with the body.

100. A device as claimed in claim 92, wherein the further body has a length of between 10 and 40 mm.

101. A device as claimed in claim 100, wherein the further body has a length of between 20 and 35 mm.

102. A device as claimed in claim 10, wherein the further body has a length of between 25 and 30 mm.

103. A device as claimed in claim 92, wherein the further body has a maximum width of between 4 and 12 mm.

104. A device as claimed in claim 10, wherein the further body has a maximum width of between 5 and 10 mm.

105. A device as claimed in claim 92, wherein in the collapsed configuration, the further body fits within a diameter of 10 mm.

106. A device as claimed in claim 105, wherein in the collapsed configuration, the further body fits within a diameter of 7 mm.

107. A device as claimed in claim 106, wherein in the collapsed configuration, the further body fits within a diameter of about 6 mm.

108. A device as claimed in claim 92, wherein the pharyngeal airway has a minimum cross-sectional area of between 4 and 9 mm² when the further body is in the expanded configuration.

109. A device as claimed in claim 108, wherein the pharyngeal airway has a minimum cross-sectional area of between 6 and 8 mm² when the further body is in the expanded configuration.

110. A device as claimed in claim 92, wherein in the expanded configuration, the further body is generally torus shaped.

111. A device as claimed in claim 110, wherein in the expanded configuration, the further body has an external diameter than is smaller than the external diameter of the body in the expanded configuration.

112. A device as claimed in claim 110, wherein in the expanded configuration, the further body has an external diameter of between 4 mm and 15 mm.

113. A device as claimed in claim 112, wherein the external diameter is between 5 mm and 12 mm.

114. A device as claimed in claim 110, wherein in the expanded configuration, the further body has an internal diameter of between 3 mm and 12 mm.

115. A device as claimed in claim 114, wherein the internal diameter is between 3 mm and 7 mm.

116. A device as claimed in claim 92, wherein the further body is a generally thin-walled structure.

117. A device as claimed in claim 116, comprising one or more fold lines to control movement of the further body between the expanded and the collapsed configuration.

118. A device as claimed in claim 92, wherein when the further body is in the collapsed configuration it is twisted.

119. A device as claimed in claim 92, wherein when the further body is in the expanded configuration, the pharyngeal airway comprises an opening towards the nasopharynx and another opening towards the mouth and tongue.

120. A device as claimed in claim 92, wherein the further body is sized to be received within a pediatric patient.

121. A device as claimed in claim 92, wherein the further body is sized to be received within an adult patient.

122. A device as claimed in claim 92, wherein the further body comprises an outer surface that is adapted to slowly release an anesthetic substance.

123. A device as claimed in claim 122, wherein the outer surface is adapted to be impregnated with the anesthetic substance.

124. A device as claimed in claim 122, wherein the further body comprises a plurality of micropores extending from the internal volume to outside the further body.

125. A device as claimed in claim 122, wherein the further body comprises a plurality of nanopores extending from the internal volume to outside the further body.

126. A device as claimed in claim 92, wherein the further body is made of a biocompatible material.

127. A device as claimed in claim 126, wherein the further body is made of plastic.

128. A device as claimed in claim 127, wherein the further body is made of a plastic out of the group of plastics comprising: polyvinyl chloride, polyethylene, PEEK, Ultem PEI, Polysulfone, polypropylene and polyurethane.

129. A device as claimed in claim 127, wherein the further body is made of silicone.

130. A device as claimed in claim 92, wherein the further body comprises a foam portion that is adapted to be compressed when the further body is in the collapsed configuration.

131. A device as claimed in claim 1, wherein in the expanded configuration, an outer surface of the body comprises one or more grooves adapted to allow fluids to drain when the outer surface is in contact with an anatomical surface in use.

132. A device as claimed in claim 92, wherein in the expanded configuration, an outer surface of the further body comprises one or more grooves adapted to allow fluids to drain when the outer surface is in contact with an anatomical surface in use.

133. A device as claimed in claim 1, wherein an outer surface of at least a portion of the guide means is adapted to slowly release an anesthetic substance in use.

134. A device as claimed in claim 133, wherein the outer surface is adapted to be impregnated with the anesthetic substance.

135. A device as claimed in claim 1, wherein the body is adapted to move between the expanded and collapsed configuration on application of an electric current.

136. A device as claimed in claim 135, wherein the body comprises one or more electro-active polymer elements and the body is adapted to move between the expanded and collapsed configuration on application of an electric current to the one or more electro-active polymer elements.

137. A device as claimed in claim 136, wherein the guide means comprises a wire adapted for connection to a selective power source at one end and to the one or more electro-active polymer elements at the other end.

138. A device as claimed in claim 92, wherein the further body is adapted to move between the expanded and collapsed configuration on application of an electric current.

139. A device as claimed in claim 138, wherein the further body comprises one or more electro-active polymer elements and the further body is adapted to move between the expanded and collapsed configuration on application of an electric current to the one or more electro-active polymer elements.

140. A device as claimed in claim 139, wherein the guide means comprises a wire adapted for connection to a selective power source at one end and to the one or more electro-active polymer elements at the other end.

141. A device as claimed in claim 1, wherein in the expanded configuration, the body is generally cylindrical in shape.

142. A device as claimed in claim 1, wherein the body comprises an inner membrane, an outer membrane and a membrane volume in between, the outer membrane comprising pore means, the body being such that when in the expanded configuration, any gel or fluid in the membrane volume is dispensed through the pore means to outside the body.

143. A device as claimed in claim 92, wherein in the expanded configuration, the further body is generally cylindrical in shape.

144. A device as claimed in claim 92, wherein the further body comprises an inner membrane, an outer membrane and a membrane volume in between, the outer membrane comprising pore means, the further body being such that when in the expanded configuration, any gel or fluid in the membrane volume is dispensed through the pore means to outside the further body.

145. NA device for maintaining an oropharyngeal airway, comprising:
a body adapted for configuration between a collapsed and an expanded configuration, wherein in the expanded configuration, the body is adapted for location in the oropharynx, to define an oropharyngeal airway and to maintain the oropharyngeal airway at least sufficiently open for breathing when the body is subjected to an external pressure, the oropharyngeal airway being substantially larger when the body is in the expanded configuration than when the body is in the collapsed configuration.

146. A device as claimed in claim 145, wherein the nasopharyngeal airway means is adapted for configuration between a collapsed and an expanded configuration, such that in the collapsed configuration, it can be readily passed through one of the nares, nasal cavity and nasopharynx and into the pharynx, and in the expanded configuration a user can breathe through the nasopharyngeal airway.

147. A device as claimed in claim 145, wherein in the expanded configuration, the further body is generally torus shaped

148. A device as claimed in claim 147, wherein the external pressure is applied by the base of the tongue.

149. A device as claimed in claim 147, wherein the external pressure is applied by the oropharyngeal wall.

150. A device as claimed in claim 147, wherein in the expanded configuration, the body is adapted to apply a pressure to one or more areas from the following group of areas:
(i) the oropharyngeal wall;
(ii) the posterior pharyngeal wall;
(iii) the base of the tongue;
(iv) the soft palate; and
(v) one or more supraglottic structures.

151. A device as claimed in claim 147, wherein in the expanded configuration, the body is adapted for location around at least a portion of the glottis, epiglottis and laryngeal opening.

152. A device as claimed in claim 151, wherein in the expanded configuration, the body is adapted for location around the glottis, epiglottis and laryngeal opening.

153. A device as claimed in claim 147, wherein the body has a length of between 10 and 40 mm.

154. A device as claimed in claim 153, wherein the body has a length of between 20 and 35 mm.

155. A device as claimed in claim 154, wherein the body has a length of between 25 and 30 mm.

156. A device as claimed in claim 147, wherein in the expanded configuration, the body is generally boot-shaped.

157. A device as claimed in claim 156, wherein in the expanded configuration, at the heel of the boot, the body has a maximum width of between 10 and 30 mm.

158. A device as claimed in claim 157, wherein in the expanded configuration, at the heel of the boot, the body has a maximum width of between 15 and 25 mm.

159. A device as claimed in claim 147, wherein in the expanded configuration, the body is generally kidney-shaped.

160. A device as claimed in claim 147, wherein the oropharyngeal airway has a minimum cross-sectional area of between 19 and 60 mm² when the body is in the expanded configuration.

161. A device as claimed in claim 160, wherein the oropharyngeal airway has a minimum cross-sectional area of between 20 and 50 mm² when the body is in the expanded configuration.

162. A device as claimed in claim 156, wherein in the expanded configuration, at the heel of the boot, the body has an external diameter of between 15 and 30 mm.

163. A device as claimed in claim 162, wherein at the heel of the boot, the body has an external diameter of between 15 and 25 mm.

164. A device as claimed in claim 156, wherein at the heel of the boot, in the expanded configuration, the body has an internal diameter of between 5 and 12 mm.

165. A device as claimed in claim 164, wherein at the heel of the boot, the body has an internal diameter of between 5 and 9 mm.

166. A device as claimed in claim 147, wherein the body is a generally thin-walled structure.

167. A device as claimed in claim 166, comprising one or more fold lines to control movement of the body between the expanded and the collapsed configuration.

168. A device as claimed in claim 147, wherein when the body is in the collapsed configuration it is twisted.

169. A device as claimed in claim 147, wherein when the body is in the expanded configuration, the oropharyngeal airway comprises an opening towards the nasopharynx and another opening towards the mouth and tongue.

170. A device as claimed in claim 147, wherein the body is sized to be received within a pediatric patient.

171. A device as claimed in claim 147, wherein the body is sized to be received within an adult patient.

172. A device as claimed in claim 147, further comprising nasopharyngeal airway means connected to the body and defining a nasopharyngeal airway, the nasopharyngeal airway being in fluid communication with the oropharyngeal airway.

173. A device as claimed in claim 172, wherein the nasopharyngeal airway means is adapted for configuration between a collapsed and an expanded configuration, such that in the collapsed configuration, it can be readily passed through one of the nares, nasal cavity and nasopharynx and into the pharynx, and in the expanded configuration a user can breathe through the nasopharyngeal airway.

174. A device as claimed in claim 172, wherein the nasopharyngeal airway means comprises a thermo-sensitive material such that at least one material property of the thermo-sensitive material changes as a function of temperature.

175. A device as claimed in claim 174, wherein the thermo-sensitive material changes shape as a function of temperature such that the nasopharyngeal airway means changes shape as a function of temperature.

176. A device as claimed in claim 172, wherein the nasopharyngeal airway means has an external diameter of between 6 and 12 mm.

177. A device as claimed in claim 172, wherein the nasopharyngeal airway means has an external diameter of between 7 and 10 mm.

178. A device as claimed in claim 172, wherein the nasopharyngeal airway has a minimum internal diameter of between 5 and 10 mm.

179. A device as claimed in claim 172, wherein the nasopharyngeal airway has a minimum internal diameter of between 6 and 7 mm.

180. A device as claimed in claim 172, wherein the nasopharyngeal airway has a minimum cross-sectional area of between 15 and 80 mm².

181. A device as claimed in claim 172, wherein the nasopharyngeal airway has a minimum cross-sectional area of between 25 and 50 mm².

182. A device as claimed in claim 172, wherein the nasopharyngeal airway means has a wall thickness of between 0.1 and 10 mm.

183. A device as claimed in claim 172, wherein the nasopharyngeal airway means has a wall thickness of between 0.3 and 0.5 mm.

184. A device as claimed in claim 173, wherein the body comprises an internal volume that is fluid inflatable such that when inflated with fluid the body is in the expanded configuration and when deflated the body is in the collapsed configuration.

185. A device as claimed in claim 184, wherein the nasopharyngeal airway means is adapted to extend from at least the nares through the nasal cavity and nasopharynx and into the pharynx.

186. A device as claimed in claim 184, wherein the nasopharyngeal airway means comprises an inflation fluid passage having a fluid port adapted for connection to a fluid supply, the inflation fluid passage being in fluid communication with the internal volume.

187. A device as claimed in claim 186, wherein when the inflation fluid passage is inflated at least a portion of the length of the nasopharyngeal airway means is in the expanded configuration and when deflated, the portion of the nasopharyngeal airway means is in the collapsed configuration.

188. A device as claimed in claim 186, wherein the fluid port comprises an airway connector.

189. A device as claimed in claim 186, wherein the body and nasopharyngeal airway means move to the expanded configuration when a fluid pressure is applied at the fluid port and to the collapsed configuration when the fluid is withdrawn through the fluid port.

190. A device as claimed in claim 186, wherein the inflation fluid passage has an internal cross-sectional area of between 0.5 and 2 mm².

191. A device as claimed in claim 186, wherein the inflation fluid passage has an internal cross-sectional area of between 1 and 1.5 mm².

192. A device as claimed in claim 186, wherein the inflation fluid passage is a lumen.

193. A device as claimed in claim 186, wherein the nasopharyngeal airway means is a dual, thin-walled structure, the inflation fluid passage provided by the volume between outer and inner walls of the dual, thin-walled structure.

194. A device as claimed in claim 186, wherein the inflation fluid passage is one or more inflation fluid sub-passages.

195. A device as claimed in claim 186, wherein the fluid supply is a pressurized fluid supply.

196. A device as claimed in claim 186, wherein an outer surface of the nasopharyngeal airway means is adapted to slowly release an anesthetic substance.

197. A device as claimed in claim 196, wherein the outer surface is adapted to be impregnated with the anesthetic substance.

198. A device as claimed in claim 196, wherein the nasopharyngeal airway means comprises a plurality of micropores extending from the inflation fluid passage to outside the nasopharyngeal airway means.

199. A device as claimed in claim 196, wherein the nasopharyngeal airway means comprises a plurality of nanopores extending from the inflation fluid passage to outside the nasopharyngeal airway means.

200. A device as claimed in claim 184, wherein an outer surface of the body is adapted to slowly release an anesthetic substance.

201. A device as claimed in claim 200, wherein the outer surface is adapted to be impregnated with the anesthetic substance.

202. A device as claimed in claim 200, wherein the body comprises a plurality of micropores extending from the internal volume to outside the body.

203. A device as claimed in claim 200, wherein the body comprises a plurality of nanopores extending from the internal volume to outside the body.

204. A device as claimed in claim 184, wherein the fluid is a gas.

205. A device as claimed in claim 204, wherein the fluid is air.

206. A device as claimed in claim 204, wherein the fluid comprises an anesthetic gas.

207. A device as claimed in claim 184, wherein the fluid is a liquid.

208. A device as claimed in claim 207, wherein the fluid is water.

209. A device as claimed in claim 207, wherein the fluid comprises an anesthetic liquid.

210. A device as claimed in claim 173, further comprising guide means connected to the body.

211. A device as claimed in claim 210, wherein the guide means is a nasopharyngeal guide means adapted to direct the body in the collapsed configuration through one of the nares, nasal cavity and nasopharynx and into the pharynx.

212. A device as claimed in claim 211, wherein the guide means is adapted to direct the body such that it does not apply a substantial pressure to the region of the turbinate at the entrance to the nasopharynx.

213. A device as claimed in claim 211, wherein the guide means is adapted to direct the body such that it does not contact the region of the turbinate at the entrance to the nasopharynx.

214. A device as claimed in claim 210, wherein the guide means is flexible.

215. A device as claimed in claim 210, wherein the guide means is semi-rigid.

216. A device as claimed in claim 210, wherein the guide means is pliable.

217. A device as claimed in claim 210, wherein the length of the guide means is bendable into different shapes.

218. A device as claimed in claim 210, wherein the guide means comprises a thermo-sensitive material such that at least one material property of the thermo-sensitive material changes as a function of temperature.

219. A device as claimed in claim 218, wherein the thermo-sensitive material changes shape as a function of temperature such that the guide means changes shape as a function of temperature.

220. A device as claimed in claim 210, wherein the guide means comprises a shape memory material and is bendable into a new shape when heated and retains the new shape when cooled.

221. A device as claimed in claim 210, wherein the guide means comprises a cord.

222. A device as claimed in claim 210, wherein the guide means has an external cross-sectional area of between 1.5 and 4 mm².

223. A device as claimed in claim 210, wherein the guide means has an external cross-sectional area of between 2 and 3.5 mm².

224. A device as claimed in claim 210, wherein the guide means comprises one or more bend regions, each bend region being adapted to move between a first angle and a second angle.

225. A device as claimed in claim 224, wherein at least one of the bend regions is adapted to move between the first angle and the second angle upon application of an electric current.

226. A device as claimed in claim 225, wherein the at least one bend region comprises an electro-active shape memory polymer element and is adapted to move between the first angle and the second angle upon application of an electric current to the electro-active shape memory polymer element.

227. A device as claimed in claim 226, wherein the electro-active shape memory polymer element is a piezo-electric polymer element.

228. A device as claimed in claim 224, wherein at least one of the bend regions is adapted to move between the first angle and the second angle upon application of light within a predetermined wavelength range.

229. A device as claimed in claim 228, wherein the at least one bend region comprises a light-induced shape memory polymer element and is adapted to move between the first angle and the second angle upon application of a light within a predetermined wavelength range to the light-induced shape memory polymer element.

230. A device as claimed in claim 225, wherein the at least one bend region comprises an electromechanical servo motor and is adapted to move between the first angle and the second angle upon application of an electric current to the electromechanical servo motor.

231. A device as claimed in claim 210, wherein the guide means comprises a series of elements connected by hinge means, and a tensioning means, the guide means being adapted to move between a first configuration in which the series of elements are disposed relative to each other in a first disposition and a second configuration in which the series of elements are disposed relative to each other in a second disposition by tensioning of the tensioning means.

232. A device as claimed in claim 231, wherein the series of elements are hollow and the tensioning means is a tensioning cord that extends through the series of elements.

233. A device as claimed in claim 210, wherein the guide means comprises a series of elements connected by hinge means, and a push means, the guide means being adapted to move between a first configuration in which the series of elements are disposed relative to each other in a first disposition and a second configuration in which the series of elements are disposed relative to each other in a second disposition by pushing of the push means.

234. A device as claimed in claim 233, wherein the series of elements are hollow and the push means extends through the series of elements.

235. A device as claimed in claim 210, wherein the guide means comprises a sheath means and a rotational member located therein, the rotational member being connected to the body at one end and extending externally from the sheath means at the other end, such that when the rotational member is rotated relative to the sheath means in a first direction the body twists to the collapsed configuration and when the rotational member is rotated relative to the sheath means in the other direction the body un-twists to the expanded configuration.

236. A device as claimed in claim 210, wherein the guide means comprises a sheath means and a push-pull member located therein, the push-pull member being connected to the body at one end and extending externally from the sheath means at the other end, such that when the push-pull member is moved relative to the sheath means in one direction the body collapses to the collapsed configuration and in the other direction the body expands to the expanded configuration.

237. A device as claimed in claim 210, wherein a proximal end of the guide means, being proximal to a patient in use, comprises a generally cone-shaped tip.

238. A device as claimed in claim 237, wherein the proximal end of the guide means is blunted.

239. A device as claimed in claim 238, wherein the proximal end of the guide means is generally frusto-conical.

240. A device as claimed in claim 237, wherein the proximal end of the guide means has a smooth outer surface.

241. A device as claimed in claim 210, wherein a proximal end of the guide means, being proximal to a patient in use, is weighted so it tends to drop down.

242. A device as claimed in claim 210, wherein a distal end of the guide means, being distal to a patient in use, comprises an anchor.

243. A device as claimed in claim 242, wherein the anchor is one or more wings that contact an outer surface of at least one of the nares in use.

244. A device as claimed in claim 242, wherein the anchor is a clip adapted for clipping onto at least one of the nares in use.

245. A device as claimed in claim 242, wherein the anchor comprises a face mask, nasal mask, head band or head gear.

246. A device as claimed in claim 242, wherein the anchor is adapted to engage the septum.

247. A device as claimed in claim 210, wherein a cross-section of a main length of the guide means fits within a diameter of 2.5 mm.

248. A device as claimed in claim 210, wherein a cross-section of a main length of the guide means fits within a diameter of 2 mm.

249. A device as claimed in claim 210, wherein the nasopharyngeal airway means comprises a nasopharyngeal guide means adapted to direct the body in the collapsed configuration through one of the nares, nasal cavity and nasopharynx and into the pharynx.

250. A device as claimed in claim 210, wherein the body, the nasopharyngeal airway means and the guide means are made of a biocompatible material.

251. A device as claimed in claim 250, wherein the guide means is made of plastic.

252. A device as claimed in claim 251, wherein the guide means is made from a plastic out of the group of plastics comprising: polyvinyl chloride, polyethylene, PEEK, Ultem PEI, Polysulfone, polypropylene and polyurethane.

253. A device as claimed in claim 250, wherein the guide means is made of silicone.

254. A device as claimed in claim 250, wherein the body is made of plastic.

255. A device as claimed in claim 254, wherein the body is made from a plastic out of the group of plastics comprising: polyvinyl chloride, polyethylene, PEEK, Ultem PEI, Polysulfone, polypropylene and polyurethane.

256. A device as claimed in claim 250, wherein the body is made of silicone.

257. A device as claimed in claim 250, wherein the nasopharyngeal airway means is made of plastic.

258. A device as claimed in claim 257, wherein the nasopharyngeal airway means is made from a plastic out of the group of plastics comprising: polyvinyl chloride, polyethylene, PEEK, Ultem PEI, Polysulfone, polypropylene and polyurethane.

259. A device as claimed in claim 250, wherein the nasopharyngeal airway means is made of silicone.

260. A device as claimed in claim 147, wherein the body comprises a foam portion that is adapted to be compressed when the body is in the collapsed configuration.

261. A device as claimed in claim 147, wherein in the expanded configuration, an outer surface of the body comprises one or more grooves adapted to allow fluids to drain when the outer surface is in contact with an anatomical surface in use.

262. A device as claimed in claim 210, wherein an outer surface of at least a portion of the guide means is adapted to slowly release an anesthetic substance in use.

263. A device as claimed in claim 261, wherein the outer surface is adapted to be impregnated with the anesthetic substance.

264. A device as claimed in claim 173, wherein in the expanded configuration, an outer surface of the nasopharyngeal airway means comprises one or more grooves adapted to allow fluids to drain when the outer surface is in contact with an anatomical surface in use.

265. A device as claimed in claim 172, wherein the body is adapted to move between the expanded and collapsed configuration on application of an electric current.

266. A device as claimed in claim 265, wherein the body comprises one or more electro-active polymer elements and is adapted to move between the expanded and collapsed configuration on application of an electric current to the one or more electro-active polymer elements.

267. A device as claimed in claim 266, wherein the nasopharyngeal airway means comprises a wire adapted for connection to a power source at one end and to the one or more electro-active polymer elements at the other end.

268. A device as claimed in claim 147, wherein the body comprises an inner membrane, an outer membrane and a membrane volume in between, the outer membrane comprising pore means, the body being such that when in the expanded configuration, any gel or fluid in the membrane volume is dispensed through the pore means to outside the body.

269. A device as claimed in claim 147, further comprising oesophageal fluid canal means defining an oesophageal fluid canal.

270. A device as claimed in claim 269, wherein a proximal end of the oesophageal fluid canal comprises an opening towards the esophagus and a distal end is located outside of the nares of the patient in use.

271. A method of creating an oropharyngeal airway, comprising the steps of:
inserting a guide means through one of the nares, nasal cavity, supraglottic region, and nasopharynx and into the pharynx to locate an expandable body in a collapsed configuration in the pharynx, adjacent the base of the tongue above the cartilaginous epiglottis; and
expanding the expandable body from the collapsed configuration to an expanded configuration to create the oropharyngeal airway through the expandable body.

272. A method of creating an oropharyngeal airway as claimed in claim 271, wherein the guide means comprises an inflation passage, the body being in fluid communication with the inflation passage and inflatable from the collapsed configuration to the expanded configuration, and the step of expanding the expandable body comprises the step of:
delivering a fluid through the inflation passage to the body to inflate the body from the collapsed configuration to the expanded configuration.

273. A method as claimed in claim 272, wherein the fluid is a gas.

274. A method as claimed in claim 273, wherein the fluid is air.

275. A method as claimed in claim 273, wherein the fluid comprises an anesthetic gas.

276. A method as claimed in claim 272, wherein the fluid is a liquid.

277. A method as claimed in claim 276, wherein the fluid is water.

278. A method as claimed in claim 276, wherein the fluid comprises an anesthetic liquid.

279. A method of creating an oropharyngeal airway, comprising the steps of:
inserting a nasopharyngeal airway means in a collapsed configuration through one of the nares, nasal cavity and nasopharynx and into the pharynx to locate an expandable body in a collapsed configuration in the pharynx substantially around the glottis, epiglottis and laryngeal opening; and
expanding the nasopharyngeal airway means and the expandable body from the collapsed configuration to an expanded configuration to create the oropharyngeal airway through the nasopharyngeal airway means and expandable body.

280. A method of creating an oropharyngeal airway as claimed in claim 279, wherein the nasopharyngeal airway means comprises an inflation passage and is inflatable from the collapsed configuration to the expanded configuration, the body is inflatable from the collapsed configuration to the expanded configuration and is in fluid communication with the inflation passage, and the step of expanding the nasopharyngeal airway means and the expandable body comprises the step of:
delivering a fluid into the inflation passage and thus also into the body to inflate the nasopharyngeal airway means and the body from the collapsed configuration to the expanded configuration.

281. A method as claimed in claim 280, wherein the fluid is a gas.

282. A method as claimed in claim 281, wherein the fluid is air.

283. A method as claimed in claim 281, wherein the fluid comprises an anesthetic gas.

284. A method as claimed in claim 280, wherein the fluid is a liquid.

285. A method as claimed in claim 284, wherein the fluid is water.

286. A method as claimed in claim 284, wherein the fluid comprises an anesthetic liquid.

287. A system for maintaining an oropharyngeal airway, comprising:
a device for maintaining an oropharyngeal airway as claimed in claim 72 or 184, further comprising a fluid port, the fluid port being in fluid communication with the internal volume; and
an inflation means connected to the fluid port and adapted to deliver a fluid into the internal volume of the body.

288. A system as claimed in claim 287, wherein the inflation means is a piston-cylinder arrangement.

289. A system as claimed in claim 288, wherein the piston-cylinder arrangement is a syringe.

290. A system as claimed in claim 287, wherein the inflation means is an electrically powered air pump.

291. A system as claimed in claim 287, wherein the inflation means is a finger operated air pump.

292. A system as claimed in claim 287, wherein the inflation means is further adapted to withdraw the fluid from the internal volume of the body.

293. A system as claimed in claim 287, wherein the fluid is a gas.

294. A system as claimed in claim 293, wherein the fluid is air.

295. A system as claimed in claim 293, wherein the fluid comprises an anesthetic gas.

296. A system as claimed in claim 287, wherein the fluid is a liquid.

297. A system as claimed in claim 296, wherein the fluid is water.

298. A system as claimed in claim 296, wherein the fluid comprises an anesthetic liquid.

299. A system as claimed in claim 287, further comprising an over pressurisation valve adapted to open and release pressure from the internal volume when the pressure is higher than a predefined pressure limit.
